SEQUENCE LISTING (11 sequences)

SEQ ID NO: 1 - protein, 5 aa, unidentified
KIGII

SEQ ID NO: 2 - protein, 5 aa, unidentified
EYNLV

SEQ ID NO: 3 - protein, 5 aa, unidentified
QYELL

SEQ ID NO: 4 - protein, 5 aa, unidentified
GCGLY

SEQ ID NO: 5 - protein, 5 aa, unidentified
DCGLF

SEQ ID NO: 6 - protein, 5 aa, unidentified
ECGLY

SEQ ID NO: 7 - protein, 5 aa, unidentified
YIGLC

SEQ ID NO: 8 - protein, 5 aa, unidentified
DIMLQ

SEQ ID NO: 9 - protein, 5 aa, unidentified
QLMLQ

SEQ ID NO: 10 - protein, 5 aa, unidentified
EFNLV

SEQ ID NO: 11 - protein, 5 aa, Una virus
EINLL

(19) United States
(12) Patent Application Publication
(10) Pub. No.: US 2025/0027072 A1
(43) Pub. Date: Jan. 23, 2025

(54) METHODS AND COMPOSITIONS RELATED TO ENGINEERED CANNABINOID RECEPTORS

(71) Applicant: BOARD OF REGENTS, THE UNIVERSITY OF TEXAS SYSTEM, Austin, TX (US)

(72) Inventors: Andrew ELLINGTON, Austin, TX (US); Elizabeth GARDNER, Austin, TX (US); Jimmy GOLLIHAR, Hewitt, TX (US); Joshua LUTGENS, Austin, TX (US); Edward MARCOTTE, Austin, TX (US); Colleen MULVIHILL, Austin, TX (US)

(21) Appl. No.: 18/712,167

(22) PCT Filed: Dec. 2, 2022
(86) PCT No.: PCT/US2022/080840

§ 371 Date: May 21, 2024

Related U.S. Application Data
(60) Provisional application No. 63/396,020, filed on Aug. 8, 2022.
(60) Provisional application No. 63/285,337, filed on Dec. 2, 2021.

(51) Int. Cl. C12N 15/10; C07K 14/705; C12N 9/22; C12N 15/11; C12N 15/90; G01N 33/68

(52) U.S. Cl. CPC C12N 15/1037; C07K 14/705; C12N 9/22; C12N 15/11; C12N 15/905; G01N 33/6845; C12N 2310/20

(57) ABSTRACT

Disclosed herein is an engineered eukaryotic cell which expresses a heterologous G protein coupled receptor (GPCR), wherein native Ste2 and/or Ste3 have been replaced with a heterologous GPCR: and further wherein native G alpha protein (Gpa1) has been replaced with a gene encoding a chimeric Gpa1. The GPCR disclosed herein can be a cannabinoid receptor. such as human cannabinoid receptor type I (CB1R). Also disclosed are methods of making the engineered eukaryotic cell. and methods of using the engineered eukaryotic cell. Also encompassed by this invention are biosensors comprising CB1R and CB2R. and methods of using them.

### CROSS-REFERENCE TO RELATED APPLICATIONS

This application claims benefit of U.S. Provisional Application No. 63/285,337, filed Dec. 2, 2021, and U.S. Provisional Application No. 63/396,020, filed Aug. 8, 2022, both of which are hereby incorporated herein by reference in their entirety.

### GOVERNMENT SUPPORT CLAUSE

This invention was made with government support under Grant no. R21 AT010777 awarded by the National Institutes of Health. The government has certain rights in the invention.

### BACKGROUND

The ability to sense and respond to environmental stimuli is a fundamental process in living organisms. In humans, the primary signal transduction machinery is the G-protein coupled receptor (GPCR), which comprises the largest protein family in the genome. Humans possess over 800 GPCRs which have evolved to sense inputs including hormones, light, neurotransmitters, ions, odors, and more (Southan 2016). GPCRs are therefore instrumental in human health, and an estimated 30% of FDA-approved drugs target this family (Hauser 2017).

Abstracted from their native physiological context, GPCRs serve as useful tools of drug discovery and metabolite screening. Compound screening for drug discovery and metabolic engineering often utilize GPCR-based readouts. The traditional workflows employ cell lines from human tissue and optical readouts of intracellular second-messenger cascades. While these methods more closely preserve the native context of the GPCR, human host cell lines are expensive to maintain and operate on slower timescales. An alternative method is to use microbial eukaryotic hosts.

In particular, Saccharomyces cerevisiae has been previously adapted to accommodate human GPCRs. These yeast have only two native, orthogonal, GPCR pathways: the pheromone response pathway and a glucose sensing system (Versele 2001). The pheromone response pathway is controlled by the Ste2/Ste3 GPCRs, which sense pheromones during yeast mating. Ligand-bound Ste2/Ste3 receptors trigger signal transduction via the G-protein heterotrimeric complex, wherein the Gpa1 Gα protein exchanges GDP for GTP, which frees the βγ heterodimer to relay the signal to a mitogen activated protein kinase (MAPK) cascade and ultimately induces gene expression via the transcription factor Ste12.

Since the pheromone response pathway is nonessential and one of the most well-characterized signal cascades ever documented, it is ideal for synthetic manipulation. In particular, the Ste2/Ste3 receptors can be replaced by human GPCRs, and the downstream signaling pathway can be sufficiently humanized such that the human GPCRs can transduce signals to trigger gene expression (Brown 2000).

While the humanized-yeast GPCR platform has been widely used in the field (Shaw 2019; Mukherjee 2015; Yasi 2021), the non-native host still confers some inherent disadvantages, wherein human receptors are often nonfunctional. Possible culprits include a failure of the receptor to properly fold, glycosylate, localize to the plasma membrane, or associate with accessory proteins. Properly formed and localized receptors can suffer from impaired gating caused by the alternative sterol composition of the yeast membrane (Lagane 2000). These and other reasons can preclude the use of some GPCRs in yeast.

One such GPCR of particular medical and industrial relevance is the Cannabinoid Receptor Type 1 (CB1R). The CB1 receptor is the primary target of the drug tetrahydrocannabinol (THC), but is also the target of endocannabinoids 2-arachadonoylglycerol (2-AG) and anandamide. Given the role of cannabinoids in the treatment of chronic pain, epilepsy, psychiatric disorders, and other conditions, there is a growing demand for next generation cannabinoid medicines. Despite the exciting prospects of a fast, inexpensive, and robust biosensor for cannabinoid ligands, there has not yet been a CB1R-yeast strain described by the community.

What is needed in the art are engineered receptors, biosensors comprising these engineered receptors, and methods of using them. These methods are useful in metabolic engineering and drug discovery, amongst other things.

### SUMMARY

Disclosed herein is an engineered eukaryotic cell which expresses a heterologous G protein coupled receptor (GPCR), wherein native Ste2 and/or Ste3 have been replaced with a heterologous GPCR; and further wherein native G alpha protein (Gpa1) has been replaced with a gene encoding a chimeric Gpa1. The GPCR disclosed herein can be a cannabinoid receptor, such as human cannabinoid receptor type I (CB1R). Further disclosed herein is a biosensor comprising the engineered eukaryotic cell.

Also disclosed herein is a method of engineering a eukaryotic cell to express a heterologous G protein coupled receptor (GPCR), the method comprising: replacing a gene encoding native Ste2 and/or Ste3 of the eukaryotic cell with a gene encoding a heterologous GPCR; and replacing a gene encoding native G alpha protein (Gpa1) of the eukaryotic cell with a gene encoding a chimeric Gpa1.

Also disclosed herein is a method of identifying a compound capable of binding to a non-naturally occurring GPCR, the method comprising: exposing a test compound to an engineered eukaryotic cell which expresses a heterologous G protein coupled receptor (GPCR), wherein native Ste2 and/or Ste3 have been replaced with a heterologous GPCR; and further wherein native G alpha protein (Gpa1) has been replaced with a gene encoding a chimeric Gpa1; evaluating whether the test compound binds to the GPCR.

Also disclosed herein is an engineered eukaryotic cell which expresses a heterologous human cannabinoid receptor type II (CB2R) protein, wherein said an N-terminal sequence of CB2R has been replaced. For example, the N-terminal sequence of CB2R can comprise an exogenous leader sequences, such as an MFα leader sequence.

Disclosed herein is a method of determining relative binding of a compound to CB1R and CB2R, the method comprising: exposing a test compound to both CB1R and CB2R; evaluating relative binding of the compound to each of CB1R and CB2R; and determining whether preferential binding occurs to CB1R or CB2R. Both CB1R and CB2R can be expressed by a cell.

Further disclosed is a biosensor for the detection of compounds which interact with CB1R, CB2R, or both, wherein the biosensor comprises both CB1R and CB2R. Either one or both of these receptors may be expressed in a cell. Either one or both of these receptors may be genetically engineered, as described elsewhere herein.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows activity between the natural ligand, 2-AG and CB2 agonists.

FIG. 2A-D shows rapid prototyping platform. A) The yeast pheromone response pathway was engineered for human GPCR signal transduction. Genes SST2 and FAR1 were knocked out. The Ste2 native GPCR was replaced by a human GPCR of interest. The G-protein GPA1 was replaced with a human-yeast chimeric G protein wherein the last 5 amino acids of the protein were from the human protein. The pheromone-inducible Fig1 gene was replaced with the fluorescent reporter ZsGreen. B) Parts for rapid prototyping of GPCRs with alternate signal sequences and pre-pro signals. C) Sixteen GPCRs from human and fungal origins were tested with and without the syn-prepro at pH 5.8 or d) pH 7.1.

FIG. 3 shows optimized trafficking. Long N-terminal domains of CB1R causes inefficient plasma membrane trafficking, even in mammalian cells. This can be exacerbated in a non-mammalian host/machinery. Modular cloning is used to try combinations of truncated receptor (d89-CB1R) and with a synthetic pre-pro signal optimized for yeast secretion.

FIG. 4 shows optimization of sterol composition. Sterols are an important part of GPCR function. The key yeast sterol is ergosterol. Cholesterol-producing yeast strains were generated which showed rescued signaling of muopiod receptors. CB1R contains a cholesterol recognition amino acid consensus sequence (CRAC).

FIG. 5A-C shows the CB1R biosensor. A) Bulk fluorescence as measured by cytometry in the JG05-2.0, WT sterol strain with 1 mM ACEA (CB1R agonist). Bars show mean ZsGreen signal (area) in a population binned for singlets only. Error bars represent standard deviation of means derived from three biological replicates. B) Dose response of ACEA with four versions of the CB1 receptor: with and without the 89-residue amino terminal truncation, and with and without the mating factor alpha pre-pro signal. Fluorescence was measured via cytometry, in which a population of singlets were binned for fluorescent response, and then the mean ZsGreen signal (area) was measured across samples in bio-triplicate. Data was normalized to background fluorescence and curves were fit via 4-parameter nonlinear regression equation (Hill slope), with a bottom constraint equal to zero. C) Population-level analysis of dose-response. Histogram represents a population gated for singlets and measured with ZsGreen signal (area).

FIG. 6 shows CB1R sensitivity to sterol composition. CB1R was tested in three contexts: 1) wild type background (ergosterol); 2) cholesterol background; 3) sterol intermediates background, by introducing human cholesterol biosynthesis genes under different strength promoters, accumulation of various intermediates can be driven.

FIG. 7A-D shows cloned version of the receptor. Four versions of the receptor were designed and cloned: (A) WT CB1R; (B) WTCB1r, yeast signal sequence (MFα); (C) Δ89-CB1R; (D) Δ89-CB1R, yeast signal sequence (MFα). The rationale is that the long N-terminal domain of CB1R causes inefficient plasma membrane trafficking (even in mammalian cells). This can be exacerbated by non-mammalian host/machinery. N-terminally truncated versions of the receptor (≢89) have been shown to improve PM trafficking without affecting ligand binding in mammalian hosts.

FIG. 8A-C shows an altered sterol background to improve CB1R signaling. (A) Replacing ergosterol biosynthesis to cholesterol requires four genes. (B) Multi-TU integration vectors to express these four genes. (C) The Tus are built combinatorially with different strength promoters at each gene.

FIG. 9A-B shows a list of 39 strains that get good coverage of the metabolome. (A) Two versions of the “base” cholesterol strain with no promoter tuning: 2 versions of the “base” cholesterol strain (one from Colleen one from Concordia) with no promoter tuning. (A) BBY1650 BY4741 fig1Δ::ENVY (gfp) sst2Δ ste2Δ far1Δ GPA1 (468-472Δ)-GNAI3 (350-354) erg5Δ::Dr.DHCR7 erg6Δ::Dr.DHCR24. WCY76 BY4741 fig1Δ::ENVY (gfp) sst2Δ ste2Δ GPA1 (468-472Δ)-GNAI3 (350-354) erg5Δ::Dr.DHCR7 erg6Δ::Dr. DHCR24. Base GPCR strain (ergosterol): JG05-2.0 BY4741 fig1Δ::ZsGreen (gfp) sst2Δ ste2Δ far1Δ GPA1 (468-472Δ)-GNAI3 (350-354) *Note all the sterol intermediate strains (CJM.49-204) are built on top of the JG05-2.0 background. (B) List of strains.

FIG. 10A-B shows that the Mu opioid receptor requires cholesterol for optimal function. (A) Cholesterol vs. ergosterol. (B) Cholesterol vs. ergosterol in signaling cells.

FIG. 11A-C shows CB1R strain sterol optimization. A) The humanized cholesterol biosynthesis pathway consisted of 4 human enzymes (DHCR24, EBP, SC5DL, DHCR7). For the sterol intermediates library, each of the 4 enzymes was combinatorically cloned with 3 promoters for different expression levels and a null spacer where the enzyme was not included. B) Δ89CB1R and C) Syn-prepro-Δ89CB1R were tested in the intermediates strains versus the WT ergosterol strain. Data was collected via cytometry. Singlets were gated into either ZsGreen positive or dark bins. Percent fluorescent cells represents the percent of singlets which were binned in the ZsGreen positive group.

FIG. 12A-D shows synthetic cannabinoid screening with a CB1R biosensor strain. (A) Overview of screening results. Negative controls DMSO (vehicle) is shown in pink circles, and inverse agonist rimonabant is shown in green triangles. Each control was included at least once per plate. 100 μM ACEA was included as a positive control and all other samples were normalized to the average fluorescence (orange line). Dotted lines represent plus or minus one standard deviation of the ACEA positive control samples. (B) Predicted non-agonists were individually examined from the larger screen. Set includes known antagonists URB447²¹, predicted antagonist WIN54,461²², non-binder HU-211²³, cannabidiol degradation product HU-311²⁴, or selective CB2 ligands A-796260²⁵, AM-1241²⁶, MDA 77²⁷. (C) Known CB1R agonists and schedule I compounds AD-PINACA, ADB-PINACA, and AB-FUBINACA and their related analogs were directly compared. Flurazepam, a known benzodiazepine, does not strongly activate the CB1R. (D) Structures of select compounds featured in (C).

FIG. 13A-E shows cannabinoid analog screening. Structural analogs of several groups of known CB1R agonists were compared. Reference agonists are shown on the right side and include (A) JWH 018, (B) JWH 398, (C) JWH 122, (D) JWH 210, and (E) PB-22.

FIG. 14A-E shows development of CB1R and CB2R biosensors. (A). The yeast pheromone response pathway was genetically modified for human receptors. Greyed out boxes represent knocked out genes. The yeast pheromone GPCR Ste2 was replaced with a human GPCR. The Fig1 pheromone response gene was replaced with a ZsGreen reporter via knock-in. (B) The parts library for rapid, modular construction of trafficking-optimized GPCRs. Syn=synthetic pre-pro, MF-α=mating factor alpha pre-pro, Ste2-SS=Ste2 signal sequence. (C) ACEA dose response with CB1R. CB1, EC₅₀=1.30 μM, 95% CI 0.80 to 2.12 μM. Δ89-CB1R, EC₅₀ 5.05 μM, 95% CI 2.64 to 9.29 μM. syn-prepro CB1R EC₅₀=0.04 μM, 95% CI 0.03 to 0.05 μM. Syn-prepro Δ89-CB1R, EC₅₀=0.15 μM, 95% CI 0.10 to 0.21 μM. (D) Dose response of endocannabinoids 2-arachidonoyl glycerol (2-AG) and N-arachidonoylethanolamine (anandamide, AEA) with CB1R. AEA, EC₅₀: 4 nM, 95% CI 2.62×10⁻⁹ to 6.92×10⁻⁹ M) than 2-AG (EC₅₀: 100 nM, 95% CI 6.60×10⁻⁸ to 1.48×10⁻⁷M). (E) Dose responses with 2-AG and AEA, CB2R. 2-AG: WT, EC₅₀=0.07 μM, 95% CI 0.0006881 to 3.70 μM; syn-prepro, EC₅₀=1.54 μM, 95% CI 0.02 to 70.9 μM; MF alpha, EC₅₀=0.16 μM, 95% CI 0.08 to 0.32 μM; Ste2, 0.15 μM, 95% CI 0.39 to 0.58 μM. AEA: WT, EC₅₀=76.9 μM, 95% CI 18.6 to 242 μM; syn-prepro, EC₅₀=7620 μM, 95% CI 630 to +infinity μM; MF alpha, EC₅₀=419 μM, 95% CI 278 to 693 μM; Ste2, 40.8 μM, 95% CI 29.3 to 56.8 μM. For all experiments, n=3. Nonlinear regression was fit as a 4-parameter logistic equation.

FIG. 15A-C shows optimization of sterol environments for CB1R and CB2R biosensors. (A) Comparison of ergosterol and cholesterol production pathways in yeast and humans, respectively, after the common intermediate zymosterol. (B) Cholesterol biosynthetic intermediates and the enzymes that act on them after zymosterol. (C) Fold change in signaling results for CB1R and CB2R in sterol-modified strains. Strains were engineered to produce cholesterol and\or cholesterol biosynthetic intermediates, and sterol content was measured using GC-MS in a previously published work reproduced here with permission. Dose responses were performed for both receptors in each strain, and fold changes of maximum signal over signal with 0 μM agonist were calculated. The agonist ACEA was used for CB1R, and AEA was used for CB2R. For all experiments, n=3. Nonlinear regression was fit as a 4-parameter logistic equation.

FIG. 16A-F shows a screen of cannabinoid receptors with a synthetic cannabinoid compound library. (A) Screen of CB1R with synthetic cannabinoid compounds at 1 μM and 10 nM concentrations. Signaling was normalized to signaling with 100 μM ACEA, set at “1”. “Dose response” compounds were those chosen for further analysis by dose response. (B) Screen of CB2R with synthetic cannabinoid compounds at 1 μM and 10nM concentrations. Signaling was normalized to signaling with 1 μM AEA, set at “1”. “Dose response” compounds were those chosen for further analysis by dose response. (C) Dose responses with FUBINACA compounds. AB-FUBINACA, EC50=0.66 nM, 95% CI 0.35 to 1.14 nM; AB-FUBINACA isomer 1, EC50-0.01 nM, 95% CI 0.01 to 0.02 nM; AB-FUBINACA isomer 2, EC50-0.23 nM, 95% CI 0.1356 to 0.42 nM; AB-FUBINACA isomer 5, EC50=15.86 nM, 95% CI 7.63 to 38.36 nM. (D) Dose responses with PINACA compounds. AB-PINACA, EC50=1.21 nM, 95% CI 0.96 to 1.52nM; ADB-PINACA, EC50-0.09 nM, 95% CI 0.05 to 0.14 nM; 5-fluoro ADB-PINACA isomer 2, EC50=2.25 nM, 95% CI 1.26 to 3.97 nM. (E) Dose responses with select synthetic cannabinoids. A-836339, EC50=16.3 nM, 95% CI 3.53 to 186 nM; A-834735, EC50=7.42 nM, 95% CI 2.12 to 22.2 nM; 5-fluoro PB-22 5-hydroxyisoquinoline isomer, EC50=216 nM, 95% CI 66.3 to 1300 nM. (F) Dose responses with select synthetic cannabinoids. 4-fluoro ADB, EC50=1.35 nM, 95% CI 0.274 to 5.03 nM; ADB-BINACA, EC50=1.15 nM, 95% CI 0.254 to 4.87 nM; AB-PINACA, EC50=107 nM, 95% CI 42.1 to 251 nM.

FIG. 17A-D shows a screen of cannabinoid receptors with a terpene library. (A) Signaling at CB1R with terpene compounds at 100 μM. Signaling was normalized to signaling with 100 μM ACEA, set at “1”. “Dose response” compounds were those chosen for further analysis by dose response. (B) Signaling at CB2R with terpene compounds at 100 μM. Signaling was normalized to signaling with 100 μM AEA, set at “1”. “Dose response” compounds were those chosen for further analysis by dose response. (C) Dose responses at CB1R with compounds that demonstrated signaling in the terpene screen over 0.25 that of the 100 μM ACEA control. (D) Dose responses at CB2R with compounds that demonstrated signaling in the terpene screen over 0.25 that of the 100 μM AEA control with the exception of atractylenolide III. Totarol, EC50=485.3 nM, 95% CI 149.4 to 1443 nM. For all dose response experiments, n=3. Nonlinear regression was fit as a 4-parameter logistic equation. For the screens, n=1.

FIG. 18A-D shows results with dual cannabinoid biosensor strains recapitulate and extend opportunities for receptor-specific targeting. (A) Synthetic cannabinoid compounds screened against CB1R and CB2R at 1 μM, normalized to the maximum signal for each receptor at this concentration. Large distances from the right and left of the plotted line connote rough bias towards CB1R and CB2R, respectively. Compounds are labeled based on if they, to our knowledge, have been characterized biochemically against one or both cannabinoid receptors. (B) Comparison of yeast experimental EC50 values with EC50 and Kᵢ values found in the literature in mammalian cells 9,48,49,58-62. When there were multiple experimental values for an agonist and receptor, the optimized sterol and leader background was chosen. (C) Rendering of previously published crystal structure CB1R PDB 6N4B50 bound to the agonist MDMB-FUBINACA. Residues expected to interact with the agonist are highlighted. Types of interactions are labeled. (D) CB2R PDB 6PT052 bound to the agonist WIN-55,212-2. Residues expected to interact with the agonist are highlighted. Types of interactions are labeled. For (C) and (D), interactions were predicted using a previously described tool63. Nitrogens are highlighted on agonists in blue, oxygens in gray, and fluorine in yellow.

FIG. 19 shows CB1R dose responses with sterol-modified strains. For all experiments, n=3. Nonlinear regression was performed with a 4-parameter logistic equation.

FIG. 20 shows CB2R doses responses with sterol-modified strains. For all experiments, n=3; data points for consecutive doses are connected with lines as a guide to the eye.

FIG. 21A-B shows signaling with control compounds at CB1R. Known agonists ADB-PINACA, AB-FUBINACA, and AB-PINACA show strong signaling at 10 nM compared to a positive control (100 μM ACEA). All other compounds (10 nM) are not predicted to activate CB1R. DMSO vehicle was also tested. (B) Synthetic cannabinoid compounds screened against CB1R and CB2R at 10 nM, normalized to the maximum signal for each receptor at this concentration. Large distances from the right and left of the plotted line connote rough bias towards CB1R and CB2R, respectively. Compounds are labeled based on if they, to our knowledge, have been characterized biochemically against one or both cannabinoid receptors.

FIG. 22 shows structure and signaling of isolated synthetic cannabinoid compounds that show biased activity. (A) Select compounds from the synthetic cannabinoid screen at both CB1R and CB2R. For all experiments, n=1.

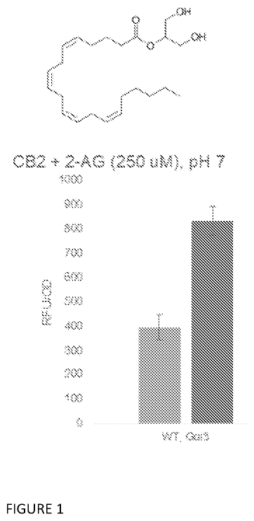

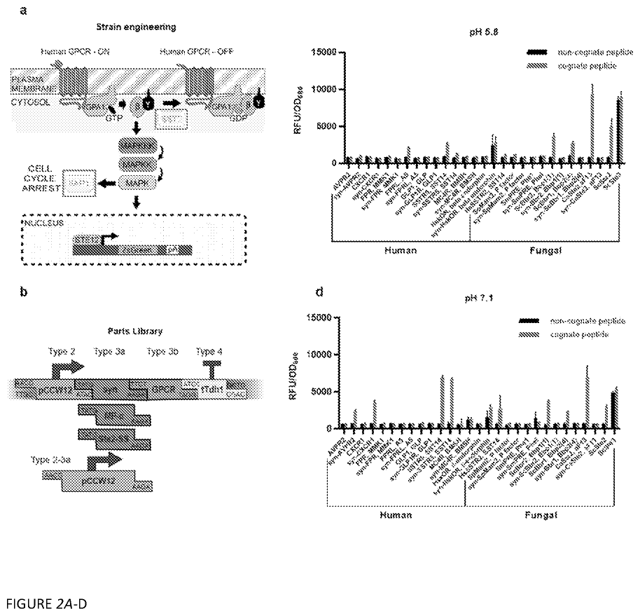

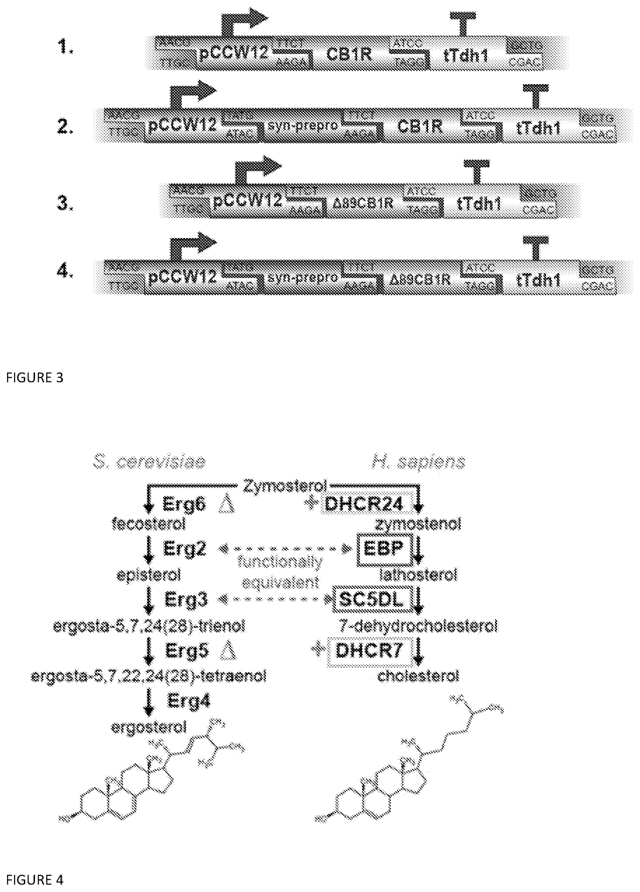

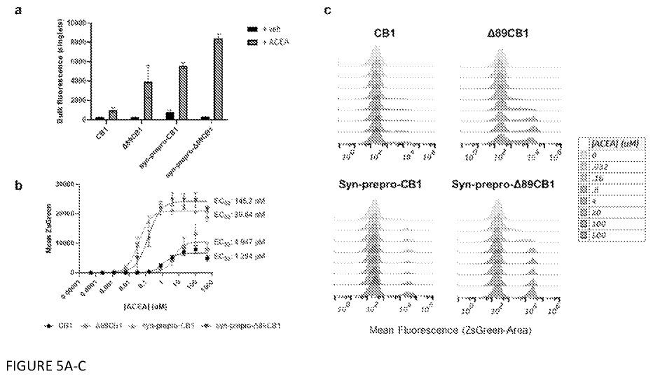

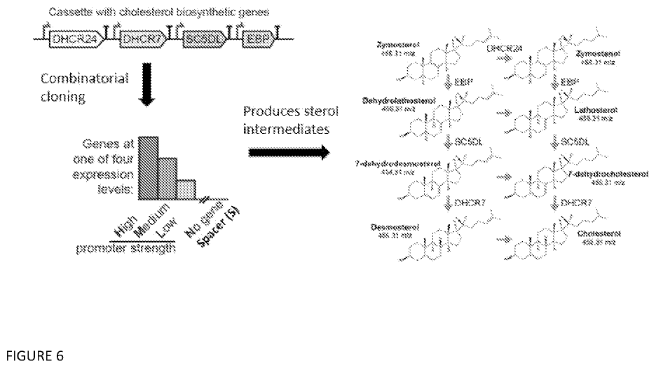

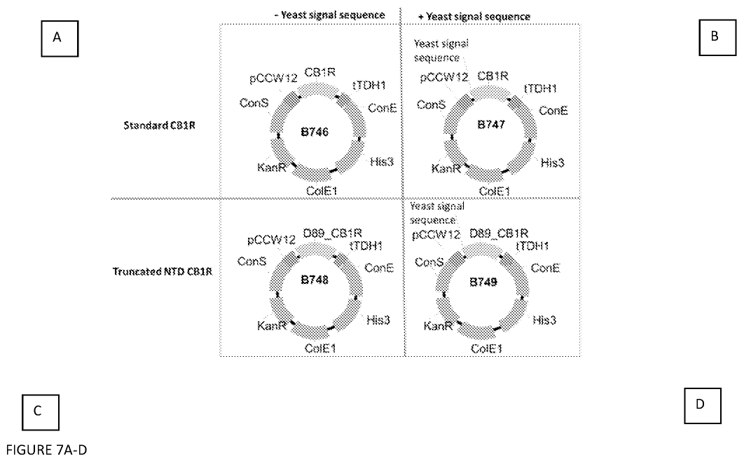

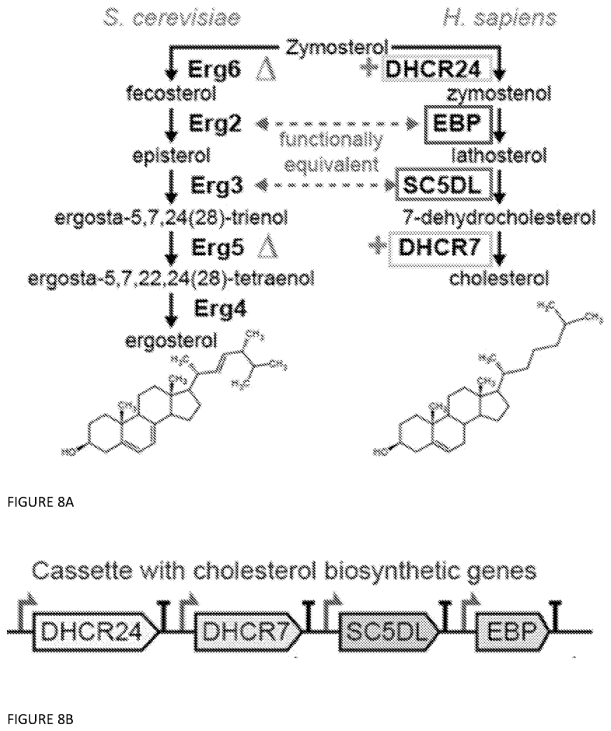

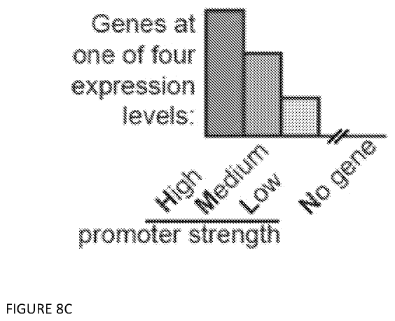

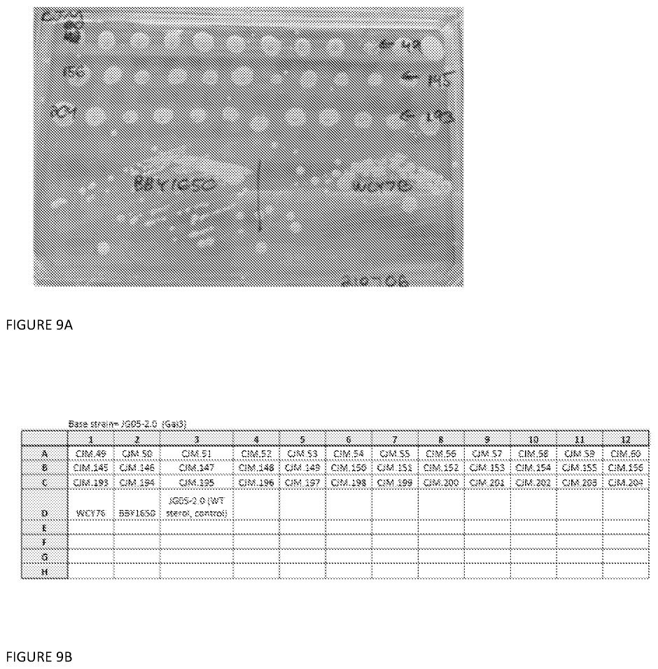

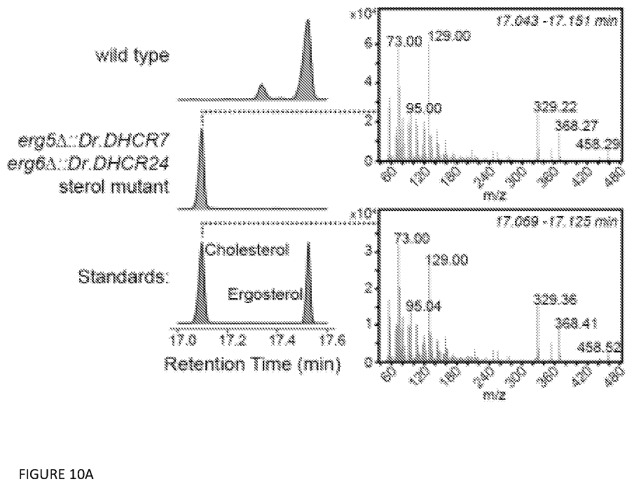

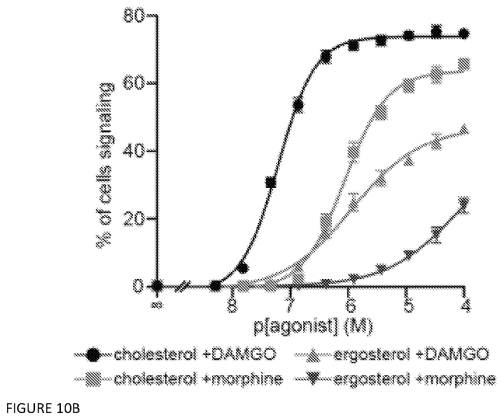

### DETAILED DESCRIPTION

### Definitions

“Biological sample” as used herein is a sample of biological tissue or fluid that contains CB1R or nucleic acid encoding CB1R protein. Such samples include, but are not limited to, tissue isolated from humans, mice, and rats, in particular, ton. Biological samples may also include sections of tissues such as frozen sections taken for histological purposes. A biological sample is typically obtained from a eukaryotic organism, such as insects, protozoa, birds, fish, reptiles, and preferably a mammal such as rat, mouse, cow, dog, guinea pig, or rabbit, and most preferably a primate such as chimpanzees or humans.

The phrase “functional effects” in the context of assays for testing compounds that modulate CB1R mediated activity includes the determination of any parameter that is indirectly or directly under the influence of the receptor, e.g., functional, physical and chemical effects. It includes ligand binding, changes in ion flux, membrane potential, current flow, transcription, G-protein binding, GPCR phosphorylation or dephosphorylation, signal transduction, receptor-ligand interactions, second messenger concentrations (e.g., cAMP, IP3, or intracellular Ca2+, in vitro, in vivo, and ex vivo and also includes other physiologic effects such increases or decreases of neurotransmitter or hormone release.

By “determining the functional effect” is meant assays for a compound that increases or decreases a parameter that is indirectly or directly under the influence of CB1R, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte CB1R expression; tissue culture cell CB1R expression; transcriptional activation of CB1R; ligand binding assays; voltage, membrane potential and conductance changes; ion flux assays; changes in intracellular second messengers such as cAMP and inositol triphosphate (IP3); changes in intracellular calcium levels; neurotransmitter release, and the like.

“Inhibitors,” “activators,” and “modulators” of CB1R are used interchangeably to refer to inhibitory, activating, or modulating molecules identified using in vitro and in vivo assays for cannabinoid transduction, e.g., ligands, agonists, antagonists, and their homologs and mimetics.

Inhibitors are compounds that, e.g., bind to, partially or totally block stimulation, decrease, prevent, delay activation, inactivate, desensitize, or down regulate activity, e.g., antagonists. Activators are compounds that, e.g., bind to, stimulate, increase, open, activate, facilitate, enhance activation, sensitize or up regulate activity, e.g., agonists. Modulators include compounds that, e.g., alter the interaction of a receptor with: extracellular proteins that bind activators or inhibitor; G-proteins; kinases; and arrestin-like proteins, which also deactivate and desensitize receptors. Modulators include genetically modified versions of CB1R, e.g., with altered activity, as well as naturally occurring and synthetic ligands, antagonists, agonists, small chemical molecules and the like. Such assays for inhibitors and activators include, e.g., expressing CB1R in cells or cell membranes, applying putative modulator compounds, and then determining the functional effects of cannabinoids on the receptor. Samples or assays comprising CB1R that are treated with a potential activator, inhibitor, or modulator are compared to control samples without the inhibitor, activator, or modulator to examine the extent of inhibition. Control samples (untreated with inhibitors) are assigned a relative CB1R activity value of 100%. Inhibition of CB1R is achieved when the CB1R activity value relative to the control is about 80%, optionally 50% or 25-0%. Activation of CB1R is achieved when the CB1R activity value relative to the control is 110%, optionally 150%, optionally 200-500%, or 1000-3000% higher.

“Biologically active” CB1R refers to CB1R having GPCR activity as described above, involved in cannabinoid interaction.

As used herein, the term “wild-type,” refers to a gene or gene product (e.g., protein) that has the characteristics (e.g., sequence) of that gene or gene product isolated from a naturally occurring source, and is most frequently observed in a population. In contrast, the term “mutant” refers to a gene or gene product that displays modifications in sequence when compared to the wild-type gene or gene product. It is noted that “naturally-occurring mutants” are genes or gene products that occur in nature, but have altered sequences when compared to the wild-type gene or gene product; they are not the most commonly occurring sequence. “Synthetic mutants” are genes or gene products that have altered sequences when compared to the wild-type gene or gene product and do not occur in nature. Mutant genes or gene products may be naturally occurring sequences that are present in nature, but not the most common variant of the gene or gene product, or “synthetic,” produced by human or experimental intervention.

The term “reporter” is used herein in the broadest sense to describe a molecular entity, a characteristic and/or property of which (e.g., concentration, amount, expression, activity, cellular post-translational modification, localization, etc.) can be detected and correlated with a characteristic and/or property of a system containing the reporter (e.g., cell, artificial cellular entity, etc.). A “reporter” may be an intrinsic (e.g., endogenous) element of the system that exhibits one or more detectable and correlatable properties, or an artificial (e.g., exogenous) element engineered or introduced into the system (e.g., artificial cellular entity), that exhibits a detectable characteristic linked to process (e.g., gene expression) or component within the system. Suitable reporters include, but are not limited to: intrinsic genes or proteins (e.g., expression, concentration, activity, or protein-protein interactions of which may be correlated to a particular stimuli), exogenous genes or proteins (e.g., expression, concentration, activity, or protein-protein interactions of which may be correlated to a particular stimuli), luciferases, a beta lactamases, CAT, SEAP, a fluorescent proteins, etc.

As used herein the term “native receptor” refers to a ligand-binding protein of a cellular entity (e.g., located on the cell surface) that is also expressed by a non-engineered ancestral cell of the cellular entity. The native receptor on the cellular entity binds a ligand recognized or bound by the native receptor of the ancestral cell.

As used herein the term “non-native receptor” refers to a ligand-binding protein of an artificial cellular entity (e.g., located on the cell surface) that is not present in/on an ancestral cell of the artificial cellular entity. The non-native receptor on the artificial cellular entity typically binds a ligand not recognized or bound by native receptors of the ancestral cell. “Non-native receptors” may be receptors that are native to another cell type, a chimera of a native receptor and a receptor native to another cell type, a mutated native receptor (e.g., having various amino acid substitutions, deletions, and/or additions), an engineered receptor (e.g., a receptor that is not native to any cell), a chimera of a native receptor and an engineered receptor, etc.

The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, optionally at least 95% pure, and optionally at least 99% pure.

“Nucleic acid” refers to deoxyribonucleotides or ribonucleotides and polymers thereof in either single-or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, or chemical means. For example, useful labels include 32P, fluorescent dyes, electron-dense reagents, enzymes (e.g., as commonly used in an ELISA), biotin, digoxigenin, or haptens and proteins for which ant or 7 can be made detectable, e.g., by incorporating a radiolabel into the peptide, and used to detect antibodies specifically reactive with the peptide).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 70% identity, optionally 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al. Nuc. Acids Res. 25:3389-3402 (1977) and Altschul et al., J. Mol. Biol. 215:403-410 (1990), respectively. Software for performing BLAST analyses is publicly available through the National Center for Biotechnology Information (which can be found on the World Wide Web at ncbi.nlm.nih.gov). This algorithm involves first identifying high scoring sequence pairs (I-ISPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The phrase “selectively associates with” refers to the ability of a nucleic acid to “selectively hybridize” with another as defined above.

By “host cell” is meant a cell that contains an expression vector and supports the replication or expression of the expression vector. Host cells may be eukaryotic cells such as yeast, insect, amphibian, or mammalian cells such as CHO, HeLa and the like, e.g., cultured cells, explants, and cells in vivo.

### General Description

Disclosed herein is a yeast strain endowed with a functional modified version of a human Cannabinoid Receptor 1 (CB1R), in which signaling via the receptor results in expression of a gene reporter. The receptor is modified from its original form such that the yeast machinery can efficiently couple to the human receptor, which is normally extremely inefficient.

There are no instances in the literature of a human CB1R expressed in yeast that can couple through the yeast GPCR signal transduction pathway (the pheromone response pathway). This is likely due to trafficking issues in which CB1R does not efficiently make it to the membrane of yeast. Also disclosed herein is an engineered cell that allows CB1R to localize and fold correctly in a way that can signal efficiently in yeast with known CB1R agonists and antagonists.

Wild-type CB1R cannot be functionally expressed in yeast using typical strategies. Provided is the advantage of increasing the fraction of CB1Rs correctly folded and at the plasma membrane of yeast. The invention can be used in a low-cost microbial drug screening platform. Uses include high throughput screening for metabolic engineering of cannabinoids, or point-of-need detection of cannabinoids in crude (or purified) extracts.

Also disclosed herein are engineered CB2Rs, as well as methods of using them and biosensors which incorporate them. Specifically, disclosed herein is a biosensor comprising both CB1R and CB2R, wherein optionally, one or both can be genetically engineered for optimized expression in a cell.

### Engineered Heterologous GPCR-Expressing Cells and Methods of Making Them

Disclosed herein is engineered eukaryotic cell which expresses a heterologous G protein coupled receptor (GPCR), wherein native Ste2 and/or Ste3 have been replaced with a heterologous GPCR; and further wherein native G alpha protein (Gpa1) has been replaced with a gene encoding a chimeric Gpa1. The GPCR disclosed herein can be a cannabinoid receptor, such as human cannabinoid receptor type I (CB1R). Also disclosed herein are methods of making and using the engineered eukaryotic cell disclosed herein.

A native GPCR can be used, along with modifications which confer desired properties. For example, the native GPCR can be comprise an N-terminus which is truncated. More specifically, when CB1R is used as the GPCR, it can localize to the mitochondria. The N-terminal can be truncated to prevent this localization. This can be done by truncating the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 amino acids. In a more specific example, the first 80 or more amino acids can be truncated. In one embodiment, the first 89 residues of the native GPCR can be truncated. A synthetic signal peptide can then be used in its stead. Synthetic signal peptides are discussed in more detail below.

In the engineered eukaryotic cell disclosed herein, several additional modifications can be made in order to optimize the functionality of the cell and the expression of the heterologous GPCR. These modifications can include replacing native genes with chimeric genes, and/or “knocking out” (rendering disabled) native genes to render them non-functional. For example, the native Gpa1 can be mutated so that it comprises 1, 2, 3, 4, 5, 6, 7, 8, 9, 10, or more amino acids from a human G-alpha protein. Examples can be found in Table 1.

TABLE 1
5 GpaI-Human Chimeras

| G-alpha | Terminal AA |
|---|---|
| Gpa1p | KIGII (SEQ ID NO: 1) |
| Gαq | EYNLV (SEQ ID NO: 2) |
| Gαs | QYELL (SEQ ID NO: 3) |
| Gαo | GCGLY (SEQ ID NO: 4) |
| Gαi2 | DCGLF (SEQ ID NO: 5) |
| Gαai3 | ECGLY (SEQ ID NO: 6) |
| Gαz | YIGLC (SEQ ID NO: 7) |
| Gα12 | DIMLQ (SEQ ID NO: 8) |
| Gα13 | QLMLQ (SEQ ID NO: 9) |
| Gα14 | EFNLV (SEQ ID NO: 10) |
| Gα16 | EINLL (SEQ ID NO: 11) |

There are various methods known to those of skill in the art for “knocking out” genes in a wild-type cell. Traditionally, homologous recombination was the main method for causing a gene knockout. Other, more contemporary methods include, but are not limited to, site-specific nucleases, zinc-fingers, TALENS, and CRISPR-Cas9. Particularly preferred for knocking out genes in the present invention is CRISPR-Cas9. The guide RNA can be engineered to match a desired DNA sequence through simple complementary base pairing/Coupled Cas9 will cause a double stranded break in the DNA. (Gaj, Thomas; Gersbach, Charles A.; Barbas, Carlos F. (2013). “ZFN, TALEN, and CRISPR/Cas-based methods for genome engineering”. Trends in Biotechnology. 31 (7): 397-405, incorporated by reference in its entirety for its disclosure concerning genome engineering). In one example, a gene can be knocked out by an in-frame stop codon followed by a barcode.

Specifically, the engineered eukaryotic cell disclosed herein can have the Sst2 gene knocked out. Furthermore, One or more cell cycle arrest genes can be knocked out, such as the Far1 gene, the Hog1 gene (stress response), and the Bar1 gene (protease).

The engineered eukaryotic cells disclosed herein can be further engineered so that they express one or more reporters. This reporter can, for example, report expression of exogenous GPCR expression. Such reporters are known to those of skill in the art. An example of one is the ZsGreen reporter. The gene for this reporter can replace the pheromone-inducible gene Fig1, for example, or other fluorescent proteins or enzymes, including thermostable polymerase.

The engineered eukaryotic cells disclosed herein can also be engineered to express one or more signal peptides, referred to herein as pre-pro, or syn-pre-pro signals, or signal sequences which are expressed along with GPCR. Engineering of these pre-pro signals can be accomplished by one of skill in the art.

In another embodiment, the cell can be further engineered with a heterologous promoter of GPCR. Engineering such heterologous promoters are known to those of skill in the art. For example, the heterologous protein can comprise a start codon.

Also disclosed herein is an engineered eukaryotic cell which expresses a heterologous human cannabinoid receptor type II (CB2R) protein, wherein said an N-terminal sequence of CB2R has been replaced. For example, the N-terminal sequence of CB2R can comprise an exogenous leader sequences, such as an MFα leader sequence. The engineered eukaryotic cell may be engineered to express an altered (non-native) sterol membrane composition. For example, the altered (non-native) sterol membrane composition can altered to increase levels of one or more cholesterol precursors. Examples of cholesterol precursors include, but are not limited to, at least one of 7-dehydrodesmosterol, desmosterol, zymosterol, zymostenol, lathosterol, or dehydrolathosterol.

### Biosensors and Platforms

Disclosed herein is a biosensor comprising the engineered eukaryotic cell described herein. The unique difficulties of engineering GPCR biosensors in yeast is described in Adeniran et al. (Adebola Adeniran, Michael Sherer, Keith E. J. Tyo, Yeast-based biosensors: design and applications, FEMS Yeast Research, Volume 15, Issue 1, February 2015, Pages 1-15, hereby incorporated by reference in its entirety). The modifications described above are key to the success of the specific engineered biosensors described herein. A wide variety of reporting systems can be used with the biosensors disclosed herein, including, but not limited to, fluorescence (GFP, RFP, YFP, ZsGreen), luminescence (Lux, Luc), colorimetric (beta-galactosidase), electrical, and growth (His3, Trp1, and Leu2). Also contemplated herein is the use of metal nanoparticles, such as gold and silver, in biosensing.

In some embodiments, the heterologous GPCR-expressing cells disclosed herein are used as biosensors within a system or device (e.g., POC system/device) configured for the detection of one or more analytes in a sample. Contemplated herein are one or more of: reagents (e.g., buffers, etc.) storage, sample purification, introduction of the sample and biosensors, mixing, reaction, signal detection, signal quantification, communication of results (e.g., on a screen, on a printer report, etc.), etc. A system/device may be of any suitable configuration for carrying out the particular detection/quantification assay. A system/device may comprise a single unit, or multiple modules (e.g., regent module, mixing module, reaction module, detection module, etc.). In particular embodiments, a system/device is configured for point-of-care applications or research applications. Exemplary systems/devices, all or portions of which may find use in embodiments herein, are described, for example, in: U.S. Pat. No. 8,697,377; WO 2014/134537; U.S. Pat. No. 7,604,592; U.S. Pat. Pub. 2013/0210652; U.S. Pat. Pub. 2014/0320807; U.S. Pat. Nos. 8,523, 797; 8,005,686; 8,283,155; 8,110,392; each of which is herein incorporated by reference in their entireties.

The biosensors described herein may find use is any suitable field. In medicine, devices/systems incorporating the GPCRs described herein find use, for example: in hospitals and medical clinics for bedside/in-room detection of biomarkers (e.g., for quick and reliable detection/diagnosis of disease, pathogen, condition, etc.); for in-the-field detection of pathogens or diagnosis; etc. In research, the biosensors herein find use, for example, in high throughput screening to search libraries of mutant proteins. These uses are discussed in more detail below. The applications/uses described herein are not limiting.

Also disclosed herein is a platform for the creation of yeast-based biosensors for GPCRs. For example, this technology takes advantage of a yeast membrane receptor that natively detects cannabinoids. The structure of the receptor is highly evolvable, making it amenable to detecting multiple ligands, both naturally and non-naturally occurring.

Further disclosed is a biosensor for the detection of compounds which interact with CB1R, CB2R, or both, wherein the biosensor comprises both CB1R and CB2R. Either one or both of these receptors may be expressed in a cell. Either one or both of these receptors may be genetically engineered, as described elsewhere herein.

### Methods of Use

Disclosed herein is a method of identifying a compound capable of binding to a non-naturally occurring GPCR, the method comprising: exposing a test compound to an engineered eukaryotic cell which expresses a heterologous G protein coupled receptor (GPCR), wherein native Ste2 and/or Ste3 have been replaced with a heterologous GPCR; and further wherein native G alpha protein (Gpa1) has been replaced with a gene encoding a chimeric Gpa1; evaluating whether the test compound (analyte) binds to the GPCR. The structure of the engineered eukaryotic cell and other optional embodiments regarding the cell itself are discussed above.

The test compound, referred to alternatively herein as the analyte, can be selected from the group comprising a polypeptide, a peptide, a small molecule, a natural product, a peptidomimetic, a nucleic acid, a lipid, lipopeptide, or a carbohydrate. Specifically contemplated herein is that the analyte is a cannabinoid which can be sensed by an engineered CB1R receptor. Cannabinoid receptors are activated by cannabinoids, generated naturally inside the body (endocannabinoids) or introduced into the body as cannabis or a related synthetic compound. More generally, a cannabinoid may be selected from among an endocannabinoid, a phytocannabinoid and a synthetic cannabinoid.

Types of endocannabinoids include, but are not limited to, arachidonoylethanolamine (Anandamide or AEA, such as anandamide, 7,10, 13, 16-docosatetraenoylethanolamide and homo-γ-linolenoylethanolamine); 2-Arachidonoylglycerol (2-AG); 2-Arachidonyl glyceryl ether (noladin ether); N-Arachidonoyl dopamine (NADA); Virodhamine (OAE or O-arachidonoyl-ethanolamine); Lysophosphatidylinositol (LPI).

Types of phytocannabinoids include, but are not limited to, tetrahydrocannabinol (Delta-9-tetrahydrocannabinol (Δ9-THC, THC) and Delta-8-Tetrahydrocannabinol (Δ8-THC)); and cannabidiol.

Synthetic cannabinoids include, but are not limited to, Dronabinol (Marinol), a Δ9-tetrahydrocannabinol (THC), used as an appetite stimulant, anti-emetic, and analgesic; Nabilone (Cesamet, Canemes); Rimonabant (SR141716); JWH-018; JWH-073; CP-55940; Dimethylheptylpyran; HU-210; HU-211; HU-331; SR144528; WIN 55,212-2; JWH-133; Levonantradol (Nantrodolum), and AM-2201. Further examples are provided in Example 3.

In the biosensors and methods of using them described herein, the analyte, or test compound, can be labeled. The biosensor can be high-throughput. Examples of high throughput systems using yeast biosensors can be found in Qiu et al. (Qiu C, Zhai H, Hou J. Biosensors design in yeast and applications in metabolic engineering. FEMS Yeast Res. 2019 Dec. 1; 19(8):foz082, herein incorporated in its entirety for its teaching regarding yeast biosensors). When the system is designed as high-throughput, a library of analytes can be used to screen for analyte-receptor interaction.

Disclosed herein is a method of determining relative binding of a compound to CB1R and CB2R, the method comprising: exposing a test compound to both CB1R and CB2R; evaluating relative binding of the compound to each of CB1R and CB2R; and determining whether preferential binding occurs to CB1R or CB2R. Both CB1R and CB2R can be expressed by a cell. Both CB1R and CB2R can be expressed by the same cell, or they can be expressed by different cells. Methods of engineering cells to express exogenous receptors is discussed herein. This can be done by using a high throughput screen, and a library of test compounds can be used. Either CB1R, CB2R, or both can genetically engineered as described elsewhere herein. A compound can preferentially bind either CB1R or CB2R, or may bind neither, or may bind both equally.

By “preferentially bind” is meant that a certain compound binds to one receptor or the other with one receptor over the other by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or 2, 3, 4, 5, 6, 7, 8, 9, or 10 fold or more, or any amount above, below, or in-between these values.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Engineered CBR1 Receptor

### Results

### Creation of Strains and Parts Design for Facile GPCR Prototyping in Yeast

To generate a testing pipeline for human GPCRs in yeast, a set of in-house base yeast strains were generated. Several genes were deleted from these strains to co-opt the pheromone response pathway for human GPCR signal transduction (FIG. 2A). Ste2, the native yeast GPCR of the MATa haplotype, was knocked out. The negative regulator SST2, a GTPase activating protein, was then deleted to maximize signal levels. Cell cycle arrest gene FAR1 was knocked out to maintain proper cell growth upon pathway activation. To generate a fluorescent reporter, ZsGreen was knocked in at the pheromone-inducible Fig1 gene. Finally, to efficiently transduce signals from the human machinery to the yeast pathway, the yeast G-alpha gene GPA1 was replaced with one of ten different GPA1-human chimeras in which the 5 C-terminal residues of the gene were replaced with that of a human G-alpha variant. These markerless genome edits were created using CRISPR-Cas9. For knockouts, an N-terminal in-frame stop codon followed by a barcode was inserted into the target region, which is designed to trigger nonsense mediated decay of the transcript. The barcode was designed to create a frameshift to prevent protein expression via translational read-through. The barcodes or knock-in genes were verified by Sanger sequencing from the genome.

Recombinant GPCRs were introduced into these yeast strains on high-copy 2μ plasmids. To rapidly clone GPCR variants, new parts were introduced to the popular MoClo yeast toolkit (Lee 2015) parts library and cloning workflow. Since heterologous expression of human GPCRs is often hampered by poor trafficking to the plasma membrane, the toolkit was expanded to include yeast-optimized pre-pro signals as a Type 3a (FIG. 2B). However, since not all receptors can accommodate N-terminal modifications, it is desirable to test both configurations. GPCRs are large genes, and costly to synthesize de novo. Therefore, the GPCR Type 3b part was designed to remain static, and the optimization parts were designed to instead “breathe” around the GPCR part. The modular N-terminal design of the GPCR necessitated a custom promoter part designated as a Type 2-3a, which includes a start codon. This part uses the same 3′ overhang as the Type 3a, which allows both versions of the receptor to be cloned from a single GPCR Type 3b part plasmid.

### Trafficking Optimization Rescues Function of Human GPCRs in Yeast

A modular cloning design was used to rapidly prototype 16 GPCRs from both human and fungal hosts. To avoid 1) purchasing many purified agonists and 2) cell wall accessibility issues, a self-signaling system was employed in which each cell could agonize its own receptor via autocrine display (Ishii 2012) of an agonist peptide. Mature peptide agonists were designed with a Flo1p C-terminal fusion designed to trap peptides between the plasma membrane and the cell wall. Each receptor was tested with its cognate peptide agonist or a non-cognate agonist (somatostatin-14 was the non-cognate agonist at fungal receptors and mating factor alpha was the non-cognate agonist for human receptors). Receptors were tested with or without the N-terminal syn-prepro. Each set of receptors was also tested in two pH conditions: pH 5.8, which is preferable for the yeast host, or pH 7.1 which is closer to the physiological pH for human cells. It was found that the syn-prepro rescued function for two of the human receptors: vasopressin receptor AVPR2 and cytokine receptor CXCR1 (FIG. 2C-D). Somatostatin receptors SSTR2 and SSTR5 did not require the pre-pro for function, underscoring that several receptors will work without further optimization. Non-human fungal receptors were tested with mating factor homologs. Several of these receptors performed worse with the syn-prepro (ScBbr2, ScBbr1, and CaSte2), possibly due to interference with the native folding or ER-insertion, especially since the S. cerevisiae host would more closely recapitulate the native context for those receptors. Another key observation was that the fungal receptors could function across the tested pHs, but the human receptors were primarily functional at pH 7.

### Engineering a Functional Human CB1R Biosensor

Having demonstrated the rapid prototyping of human and fungal GPCRs rescuing function of several receptors, the focus was on the CB1 receptor, due to its medical and industrial importance. Initially, the WT receptor showed low (<4-fold) function (FIG. 5A), likely owing to defects in plasma membrane localization. CB1 is known to have a long N-terminal domain, which can block co-translational insertion into the ER (Anderson 2003). Further, in humans the N-terminal domain is known to direct these receptors to partially localize to the mitochondria (Hebert-Chatelain 2016), but it is unclear if this is maintained in yeast. To address these concerns, both a full length and an N-terminally truncated version of the CB1 receptor was created, and each one was tested with and without the syn-prepro. The syn-prepro alone improved the on/off ratio to approximately 7-fold, and the truncated receptor alone had a larger effect with a 15-fold on/off ratio. The strongest version of the receptor included both, and had a 28-fold on/off ratio. To examine the sensitivity of the receptors, a dose response was performed (FIG. 5B). The strongest signalers were also the most sensitive: the syn-prepro-Δ89-CB1R had an EC₅₀ of 145.2 nM (n=3, 95% C.I.=0.1040 to 0.2003), and the syn-prepro-CB1 EC₅₀ was 36.64 nM (n=3, 95% C.I.=0.03163 to 0.04992), whereas the WT-CB1R and the Δ89-CB1R had EC₅₀ in the low μM range. Notably the Δ89 modification caused a 3.6-3.8-fold drop in sensitivity compared to the full-length counterparts. This can indicate a role in ligand-binding or signal transduction from the missing N-terminal fragment. This modification also consistently showed a higher total fluorescence, which can be caused by a higher fraction of signaling cells in the population (FIG. 5C).

### Native Yeast Sterols Augment CB1R Function Relative to Cholesterol and its Intermediates

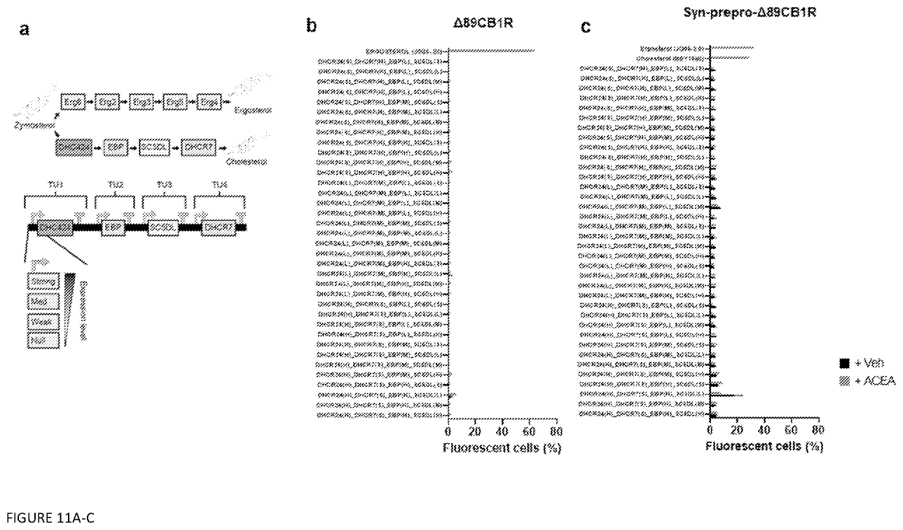

In addition to optimizing the trafficking of the receptor, other improvements were explored. One major difference between yeast cells and human cells is their sterol composition, in which the primary sterol of human membranes is cholesterol, and yeast instead produce ergosterol. Cholesterol is known to be necessary for the function of some GPCRs (Gimpl 2016) and ˜40% of GPCR PDB structures have co-crystalized with cholesterol (Sarkar 2021). Notably, CB1R is known to contain cholesterol recognition amino acid consensus (CRAC) motifs, and has been demonstrated to be negatively regulated by cholesterol (Oddi 2011). GPCR-yeast strains with a cholesterol biosynthesis pathway were created, and improved function of several human GPCRs including the mu opioid receptor was shown (Bean 2021). In addition to the cholesterol producing strain, a library of 249 sterol intermediate strains were created, where human cholesterol biosynthesis genes were placed under the control of different strength promoters and cloned combinatorically (FIG. 11A). These strains were previously evaluated by mass spectrometry and shown to have unique metabolic profiles. From this starting strain library 38 strains were selected for coverage of the metabolic space. The Δ89-CB1R and syn-prepro-Δ89-CB1R were evaluated in these strains versus the ergosterol strains. Compared to ergosterol, cholesterol-producing yeast had slightly attenuated signaling (FIG. 11B), which can indicate that the negative regulation effect of cholesterol is not extended to ergosterol. This would also imply that CRAC motifs are relatively specific to cholesterol instead of ergosterol. Strikingly, all of the intermediate strains were nonfunctional (FIG. 11C). This result stands in opposition with studies showing that depletion of cholesterol improves CB1R signaling (Bari 2005) since the intermediate strains have no cholesterol. However, in these studies about a third of the initial cholesterol remained after depletion. This result shows that cholesterol has a dual role in CB1R function, wherein cholesterol is required to maintain physical properties of the plasma membrane (Hung 2016), but simultaneously exerts a negative regulatory effect presumably through direct, selective binding of the CRAC motif.

### Methods

### GPCR Signaling Assay

GPCR-yeast strains were grown in SD-media overnight to saturation. In the morning, cultures were diluted 1:10 into 500 μL SD-media buffered to pH 7.1 with 100 mM MOPSO. Ligands were added to the media. ACEA was added at 1 mM unless otherwise stated. Cultures were grown in a deep-well grow block at 30° C. with shaking for 8 hours. Cells were washed in 50 mM Tris buffer and analyzed via cytometry with a Sony SA3800 at 10,000 events per well.

### Discussion

Cannabinoids are of great interest due to their growing therapeutic role in a variety of diseases. However, drug development of these compounds is hampered by psychoactive side effects primarily caused by CB1Rs expressed in the central nervous system. CB2 receptors, in contrast are considered non-psychoactive, and are expressed largely outside of the nervous system in immune cells where they modulate inflammation (Turcotte 2016). Therefore, it is of great interest to test emerging drug candidates against both the CB1R and the CB2R. Furthermore, an open question in cannabinoid pharmacology is the potential therapeutic role of biased agonists, or ligands that can stimulate alternative intracellular signaling pathways through the same receptor (Patel 2021; Diez-Alarcia 2016). This novel CB1R strain is useful for testing this. CB1 and CB2 receptors can be tested with different G-alpha proteins where signal transduction “signatures” could be acquired for individual drugs of interest. Detailed signaling profiles for cannabinoids could help researchers separate therapeutic benefits from side effects.

Beyond therapeutics, the recreational cannabis industry is also gaining enormous traction. Unlike the pharmaceutical industry, these facilities often lack even basic means for testing purified compounds or plant extracts against human receptors. Yeast are inexpensive to grow and exceptionally robust to a range of environments. Previously, point-of-need devices based on yeast-GPCR biosensors have been invented (Ostrov 2020) but there is not yet one for CB1R-mediated detection. Such a device could be useful for quality control and strain characterization at cannabis facilities.

The yeast-CB1R strain can also be used in the biosynthesis of cannabinoids. Previously, the complete biosynthesis of cannabinoids and unnatural analogues has been shown in yeast (Luo 2019). However, the characterization of products was performed with traditional LC-MS, which limits the throughput of pathway engineering. In contrast, cannabinoid biosynthesis pathways can be screened or selected in massive parallel (10⁹) for a desired pharmacological phenotype and product titer.

The disclosed CB1R-yeast strain was directly enabled by rapid-prototyping method. It was found that this method was able to rescue a number of human GPCRs beyond the CB1R. For non-native fungal GPCRs, trafficking modifications were often detrimental for function. A parts library was designed to rapidly test receptor sensitivity to trafficking domains, and it was found that the syn-prepro was sufficient to rescue many GPCRs. CB1R required additional modifications including an N-terminal truncation to increase plasma membrane trafficking. This provides the first evidence that CB1R can be directed to the yeast mitochondria in the same way that occurs in human host cells. CB1R was also tested in a variety of humanized sterol backgrounds, where it was found that cholesterol negatively regulated signaling capabilities relative to the yeast sterol ergosterol. Ultimately it is believed that a holistic approach to optimization of human GPCRs in yeast can be used for other receptors. By optimizing recalcitrant receptors, the field can bring more GPCR-yeast biosensors into existence, especially those of extreme therapeutic and recreational relevance such as the CB1R.

### Example 2: High-Throughput Drug Screening With the CB1R Biosensor

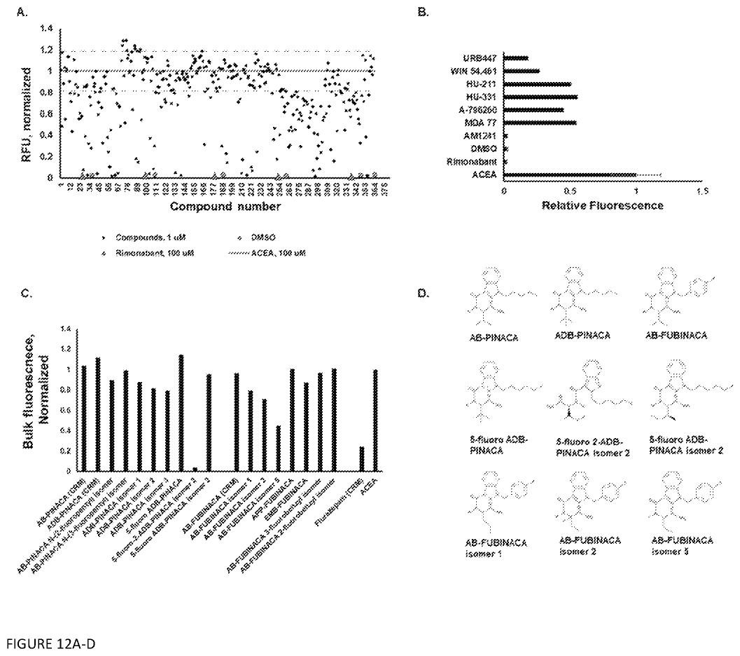

One of the main advantages of the yeast-based biosensor is its ease of use for high-throughput screening. To demonstrate the strain's capabilities for drug discovery, a compound library of over 300 synthetic cannabinoids was screened in the wild type ergosterol strain. As a negative control, DMSO or the inverse agonist rimonabant was included into each plate. The biosensor screening assay showed that a large proportion of the compounds were active at CB1 receptors (FIG. 12A). To validate the results, known and predicted agonists and antagonists of the CB1R were examined individually. First, compounds known or predicted not to strongly activate CB1Rs were tested including known antagonists URB447, predicted antagonist WIN54,461, non-binder HU-211, cannabidiol degradation product HU-311, or selective CB2 ligands A-796260, AM-1241, MDA 77. All of these compounds showed at most approximately half the relative fluorescence of the ACEA positive control, which indicated that the assay was able to differentiate agonists from non-agonists (FIG. 12B). Notably, all test compounds were delivered at 1 μM which is orders of magnitude in excess of typical EC50 values of agonists, which are generally in a nM range. Therefore, even compounds with relatively low affinity may show some partial activity in this assay.

The compound library also included several known agonists and their analogs. For example, AB-PINACA, ADB-PINACA, and AB-FUBINACA are all structurally related Schedule I controlled substances found in synthetic cannabis products. It was determined that these compounds indeed exhibited maximal fluorescence in the assay compared to 100 μM ACEA, but the Schedule I benzodiazepine Flurazepam did not show strong signal. (FIGS. 12C-D). In comparison, most of the AB-PINACA analogs also showed relatively strong signaling at 1 μM, except the 5-fluoro-2-ADB-PINACA isomer 2 (FIG. 12D). The most striking structural difference of this compound is the position of the pentyl fluoride on the diazole ring, which sterically precludes rotation of the carboxamide and subsequent positioning of key interacting heteroatoms relative to the receptor. As a result, the key moiety (the heteroatom of the carbamoyl) cannot position correctly for receptor activation. While the ring structure is an important feature of this class of molecules, the precise 3-dimensional positioning of the carbamoyl moiety can facilitate robust receptor activation through carefully coordinated contacts, whether polar or H-bonding.

The AB-FUBINACA isomers also illuminated structure activity relationships. In addition to the positioning of the carbamoyl relative to the diazole ring, this series of compounds illuminates the importance of nonpolar contacts in agonist recognition. AB-FUBINACA isomer 1 showed the greatest activity of all the isomers, followed by isomer 2, and finally isomer 5 had the least GPCR activation. Here longer aliphatic groups branching from the bridgepoint carbon of both amides results in greater activation. The length of the aliphatic chain is the determinant in this series, where nonpolar, van der Waals interactions are key for receptor binding.

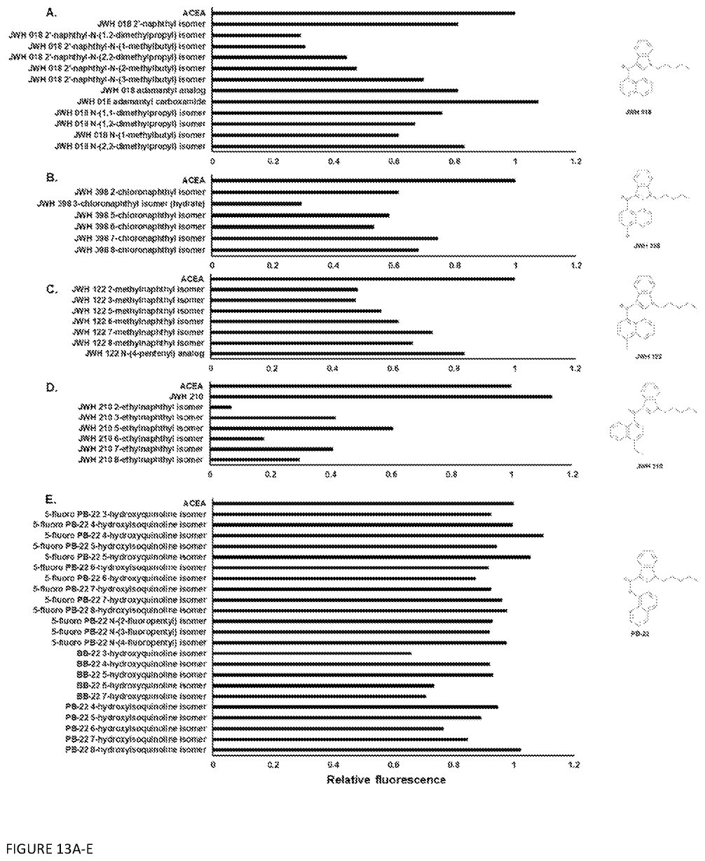

Other compound analog groups were also examined. JWH 018 is a highly potent synthetic cannabinoid that is commonly found in recreational herbal blends known as “K2/spice”. Other structurally related cannabinoids include JWH 398, JWH 122, JWH 210, and PB-22. Analogs of each of these compounds were tested in groups. Overall, each group of analogs was fairly active at 1 μM, where the PB-22 analogs were the most potent and the JWH 210 had the least active analogs (FIG. 13). In general, these compounds show a broad selectivity of the CB1R, and provides several useful starting points for further drug development.

### Example 3: Humanized CB1R and CB2R Yeast Biosensors Enable Facile Screening of Cannabinoid Compounds

Yeast expression of human G Protein Coupled Receptors (GPCRs) can be used as a biosensor platform for the detection of pharmaceuticals. The Cannabinoid receptors type 1 and 2 (CB1/2R) are of particular interest, given the cornucopia of natural and synthetic cannabinoids being explored as therapeutics. Disclosed here is that engineering the N-terminus of CB1R allows for efficient signal transduction in yeast, and that engineering the sterol composition of the yeast membrane optimizes CB2R performance. Using the dual cannabinoid biosensors, large libraries of synthetic cannabinoids and terpenes, for example, can be quickly screened to elucidate known and novel structure-activity relationships, including compounds and trends that more selectively target each of the two receptors. The biosensor strains offer a ready platform for evaluating the activity of new synthetic cannabinoids, monitoring drugs of abuse, and developing molecules that target the therapeutically important CB2R receptor while minimizing psychoactive effects.

### Introduction

GPCRs of particular medical and industrial importance are the Cannabinoid Receptors, Type 1 (CB1R) and Type 2 (CB2R). The most abundant GPCR in the brain (Irving et al. 2008), CB1R is activated by the psychoactive drug tetrahydrocannabinol (THC), but is also the target of endocannabinoids 2-arachadonoylglycerol (2-AG) and anandamide (AEA) (Zou et al. 2018). These neurotransmitters exist as lipid precursors embedded in cell membranes where they are cleaved by lipases and liberated for receptor activation (Zou et al. 2018). Endocannabinoid regulation via CB1R is implicated in neuronal excitability, where retrograde transmission of endocannabinoids from postsynaptic cells activates CB1R on presynaptic neurons and negatively regulates presynaptic neurotransmission via the Gaio pathway (Lu et al. 2016). CB1R dysregulation is, in turn, associated with schizophrenia (Ranganathan et al. 2016). In contrast to CB1R, the second of the two cannabinoid receptors, CB2R, is primarily expressed in leukocytes, where it regulates immune function. CB2R activation is broadly associated with an anti-inflammatory effect where CB2--mice exhibit increased leukocyte recruitment and inflammatory marker production (Turcotte et al. 2016). CB2R is not linked to psychoactivity and is a promising drug target for inflammatory diseases including arthritis, atherosclerosis, and inflammatory bowel disease (Turcotte et al. 2016).

Synthetic cannabinoids have been developed to elicit a response from one or both cannabinoid receptors. For decades these drugs have been sold illicitly to consumers looking for similar psychoactive effects as THC. They are quite popular, being the second most-used illegal substance by young adults (Tai et al. 2014). Unlike THC, these compounds are typically not identified in conventional drug screens. Many synthetic cannabinoids are much tighter binders to cannabinoid receptors than THC, and are often sold as mixtures uncharacterized for human use (Tai et al. 2014). Together, the high potency and lack of regulation of these compounds have led to many cases of adverse effects from recreational use, including acute psychosis, seizures, dependence and death (Tai et al. 2014; Adams et al. 2017; Lobato-Frietas et al., 2021; Tokarczyk et al. 2022). Governments have attempted to regulate these compounds, but regulation remains a challenge as new compounds and analogs of existing ones are created frequently that evade restriction (Tai et al. 2014).

Given the role of cannabinoids in the treatment of chronic pain, epilepsy, and psychiatric disorders, there is a growing demand for next generation cannabinoid medicines. Ideal therapeutic candidates should activate CB2R while avoiding potent activation of CB1R and triggering subsequent psychoactive effects. Discrimination between the two receptors is challenging, as CB1R and CB2R share a high degree of sequence similarity, including almost identical binding pockets (Hua et al. 2020).

Cannabinoid biosensor yeast strains have the potential to serve as a rapid, inexpensive, and robust screening platform. Unfortunately, they have not yet enabled facile comparisons between CB1R and CB2R activation. Herein, the engineering of CB1R and CB2R yeast biosensors by combining synthetic biology approaches that target receptor trafficking and membrane composition is shown. Using these optimized strains, more than 400 synthetic cannabinoids and terpenes were screened, known effectors were characterized, and unknown functions of cannabinoids were discovered, including analogs of controlled drugs of abuse. The dual cannabinoid biosensors provide a rapid functional screen that can be used to readily rationalize structure-activity relationships at each receptor, and should accelerate the development of safe cannabinoid therapeutics into the future.

### Results and Discussion

### Engineering Cannabinoid Receptor Function in Yeast

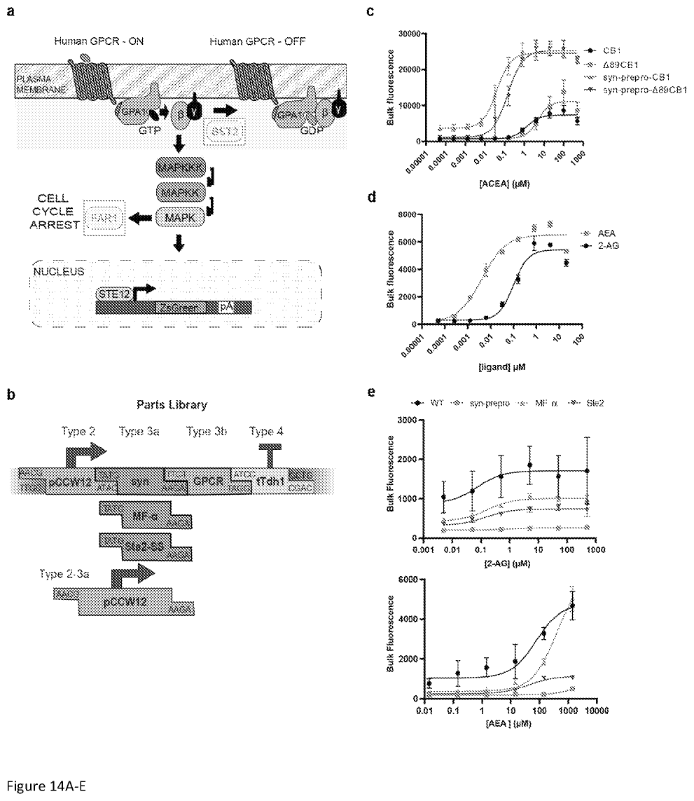

While functional human GPCRs have previously been expressed in yeast, this had proved challenging for CB1R. To enable function, Ste2 (the native yeast GPCR of the MATa haplotype), the negative regulator SST2 (a GTPase activating protein), and FAR1, which regulates cell cycle, were deleted in a manner consistent with prior efforts (Lengger et al. 2019). To efficiently transduce signals from the human machinery to the yeast pathway, the yeast 5 C-terminal residues of the G-alpha gene GPA1 were replaced with the sequence of the G_{αi3} human G-alpha variant as previously described (Brown et al. 2000). ZsGreen1 was used to report on the activity of the pathway, integrating it into the genome in place of the pheromone-inducible Fig1 gene (FIG. 14A).

Initially, the wild-type, leaderless CB1R showed low (<4-fold) signaling with the synthetic cannabinoid ACEA (FIG. 14C), which we speculated might be due to defects in plasma membrane localization. To improve basal function, we modified the popular MoClo yeast toolkit parts library (Lee et al. 2015) to include yeast-optimized pre-pro signals (designated Type 3a) after the Type 2 promoter and before the Type 3 GPCR, yielding seamless fusions (FIG. 14B). Human CB1R also has a long N-terminal domain that can block co-translational insertion into the endoplasmic reticulum, thus partially re-directing the receptor to localize in the mitochondria (Hebert-Chatelain et al. 2016). To better specify transport, a N-terminally truncated version of the CB1 receptor was created, both with and without the syn-prepro secretion sequence. The syn-prepro modification alone improved activation to approximately 7-fold, while the truncated receptor showed 15-fold activation; both modifications together yielded 28-fold activation. However, this improved dynamic range came at a cost: the syn-prepro-Δ89-CB1R was ultimately found to have an EC₅₀ of 147 nM, a 3.8-fold drop in sensitivity compared to the full-length, syn-prepro construct (FIG. 14C). This tradeoff may be due to an alteration in conformational equilibria in the deletion variant, since syn-prepro-Δ89-CB1R also showed a higher fraction of signaling cells in the population (FIG. 5C). The CB1R biosensor strain was also benchmarked with the endocannabinoids 2-AG and AEA (FIG. 14D), and was found to have sensitivities (AEA EC₅₀ of 4 nM; 2-AG EC50 of 100 nM) in accord with previously published values (Smith et al. 2015). The dramatic functional improvement achieved by the combination of N-terminal truncation and addition of a synthetic pre-pro leader highlights that proper receptor trafficking can be an issue when expressing GPCRs in yeast heterologously. Since dynamic range was likely to be of greatest utility for screens, especially with compounds with otherwise unknown affinities, we chose the combination of syn-prepro-Δ89-CB1R with ACEA to serve as the benchmark for further studies.

Unlike CB1R, CB2R is functional in yeast in its native form (Miettinen et al. 2022; Shaw et al. 2022). To further optimize function,, concordant with previous findings in mammalian cells that CB2R is more sensitive to 2-AG than AEA (Gonsiorek et al. 2000). Again, since dynamic range was of greatest import for maximizing screening capabilities, the MFα-CB2R construct and AEA were selected moving forward.

In addition, despite the fact that human GPCRs typically work in yeast at neutral pH (Ishii et al. 2012; Bean et al. 2022; Kapolka et al. 2020), CB2R showed prohibitively strong constitutive signaling at pH 7.1 (a phenomenon also previously noted with the heterologously expressed serotonin receptor, Lengger et al. 2022). When the biosensor was assayed at pH 5.8, greatly reduced background with both AEA and 2-AG were observed. Therefore, all compounds were subsequently screened with the CB2R biosensor strain at pH 5.8.

### Refactoring Sterol Composition Can Enhance Cannabinoid Receptor Activity

Cholesterol is necessary for the function of many GPCRs (Gimpl et al. 2016) (with ˜40% of GPCR PDB structures being co-crystallized with cholesterol, Sarkar et al. 2022), and providing cholesterol or intermediate metabolites to yeast can dramatically impact receptor function (Bean et al. 2022). Since CB1R is known to contain cholesterol recognition amino acid consensus (CRAC) motifs (Oddi et al. 2011), it was hypothesized that introducing the cholesterol biosynthetic machinery into CB1R-and CB2R-expressing biosensor strains could further improve receptor function.

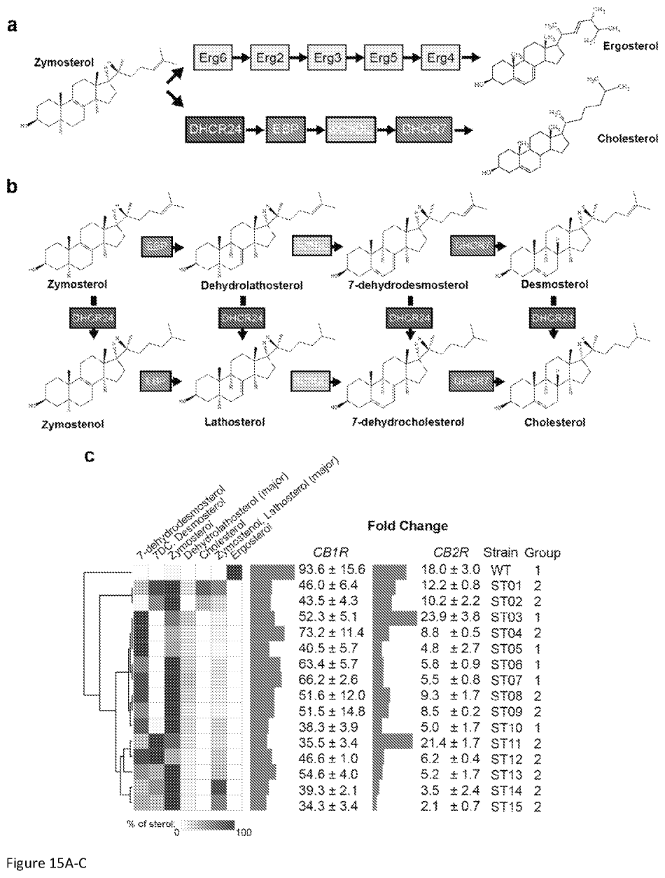

Yeast and human sterol biosynthesis share a common zymosterol precursor that can be converted to ergosterol through a five enzyme pathway or to cholesterol through a four enzyme pathway (FIG. 15A). The four-enzyme pathway produces seven metabolites (including cholesterol) in addition to zymosterol (FIG. 15B). Multiple yeast strains were previously constructed that produced varying levels of these seven metabolites (Bean et al. 2022).

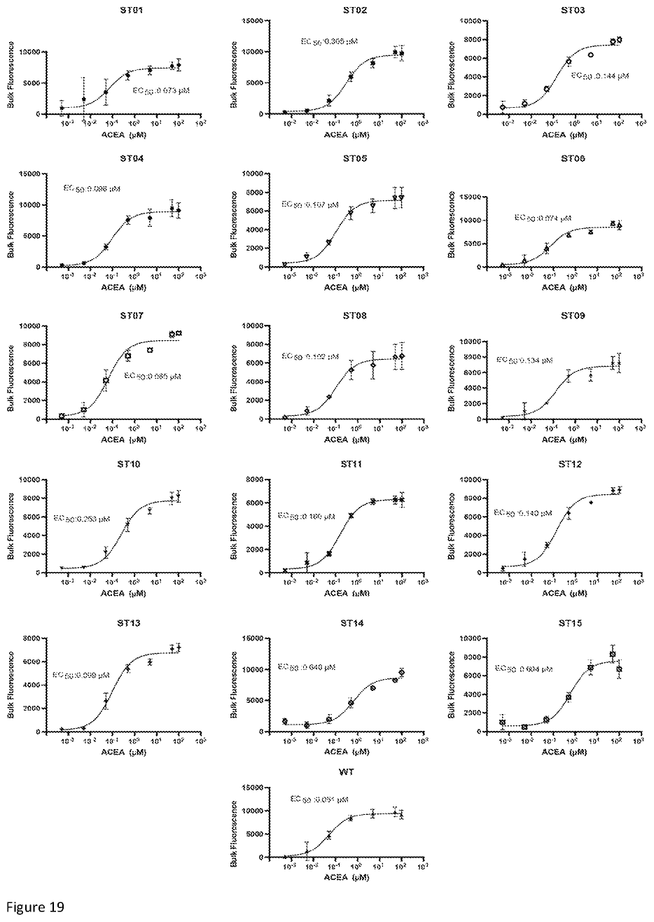

The syn-prepro-Δ89-CB1R expression construct was transformed into fifteen strains selected to cover the metabolic space. Previously, cholesterol was shown to negatively regulate CB1 (Oddi et al. 2011) receptors, although it was unclear if ergosterol would have the same effect, as it differs by two double bonds and a methyl group. Dose-responses with ACEA yielded a striking pattern in which all cholesterol intermediates were found to have a deleterious effect on CB1R signaling compared to ergosterol only (FIG. 15C), with strains containing cholesterol or any of its intermediates exhibiting lower sensitivities and dynamic ranges in signaling (FIG. 19). The wild type was thus selected, ergosterol-producing strain for subsequent studies, as it showed both the highest fold-change and most sensitive signaling (EC₅₀ of 51 nM, FIG. 19).

Similarly, MFα-CB2R was transformed into the fifteen strains with distinct sterol environments and determined dose-responses using AEA (FIG. 15C). Unlike CB1R, the literature is mixed regarding cholesterol's effect on CB2R function: Prior studies have shown that depletion of cholesterol has no effect on 2-AG binding to CB2R, but also that cholesterol increases constitutive activity of the receptor and alters ligand binding profiles (Bari et al. 2006; Yeliseev et al. 2021).

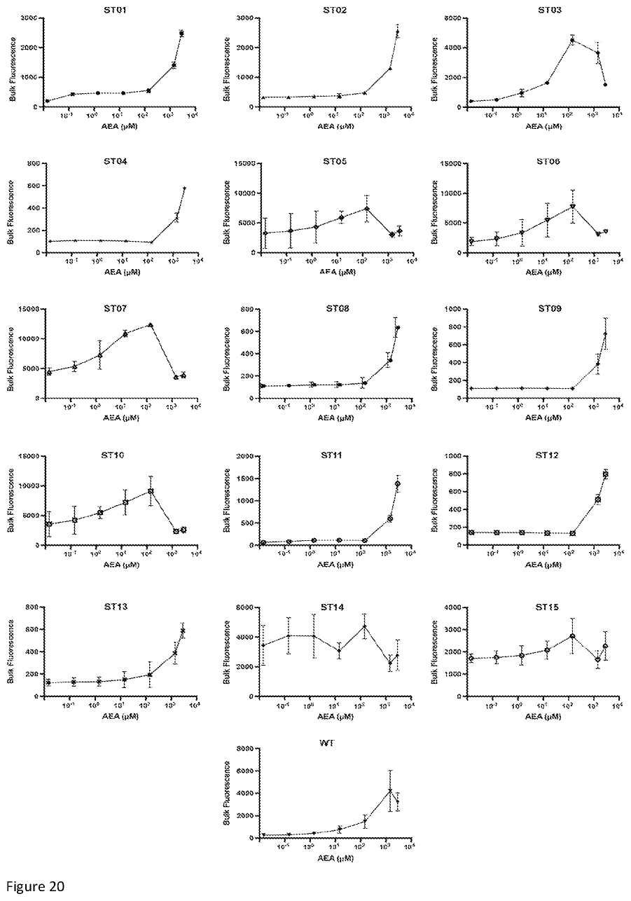

Some of the strains tested (ST01, ST02, ST04, ST09, ST11, ST12, and ST13) showed low responsivity until maximal concentrations of AEA were added, making EC50determinations impossible (FIG. 20). Other strains (ST03, ST05, ST06, ST07, and ST10) proved more sensitive to AEA than the wild-type with ergosterol, but showed a dip in signaling at the highest AEA concentrations (FIG. 20). Interestingly, this latter phenomenon has been observed previously with other CB2R agonists in yeast (Miettenen et al. 2022), and is consistent with known functional interactions between cholesterol and cannabinoids. It was suspected that compound interactions at high concentrations with cholesterol and cholesterol-like membrane components may alter membrane fluidity or other features in a way that is deleterious to receptor function (Scala et al, 2018).

For CB2R, a clear distinction between the membrane compositions of the two classes of strains was not observed, nor was it obvious which specific set of membrane components led to improved signaling, although there was a general trend between reduced signaling and the presence of the cholesterol-adjacent compounds 7-dehydrocholesterol and desmosterol. This lack of clarity emphasizes the need for a screening-based approach to membrane engineering, as shown with other human GPCRs (Bean et al. 2024). For subsequent experiments, the ST03 strain was used as it showed the highest dynamic range and was also sensitive at lower concentrations of AEA, with a greater signal at lower concentrations of AEA compared to background than the wild type ergosterol-producing strain (FIG. 20).

### High-Throughput Drug Screening of Cannabinoids With Dual Cannabinoid Receptor Biosensor Strains

The availability of two biosensor strains with optimized cannabinoid receptor function immediately presents an opportunity to determine the relative activities of a variety of cannabinoids and other compounds. A compound library of over 300 synthetic cannabinoids was screened against both strains; as a negative control, DMSO or the inverse agonist rimonabant was included on each plate analyzed by flow cytometry. The compound library included known characterized agonists and or structurally similar compounds; for example, AB-PINACA, ADB-PINACA, and AB-FUBINACA are all structurally related Schedule I controlled substances found in synthetic cannabis products. All test compounds were delivered at 1 μM, orders of magnitude in excess of typical EC50 values for agonists, in order to identify even low affinity interactions. To better identify highly active compounds, assays were also carried out at 10 nM concentrations.

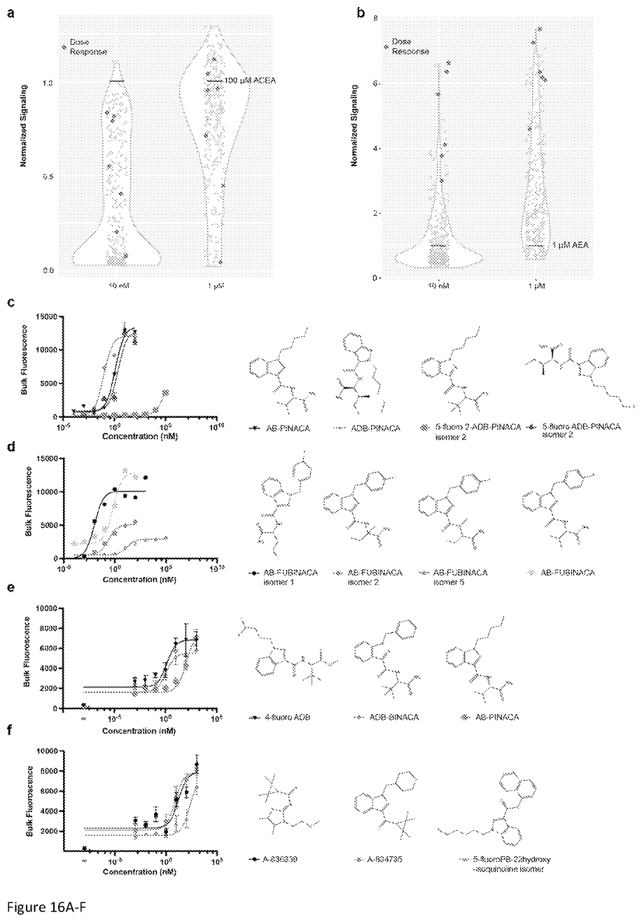

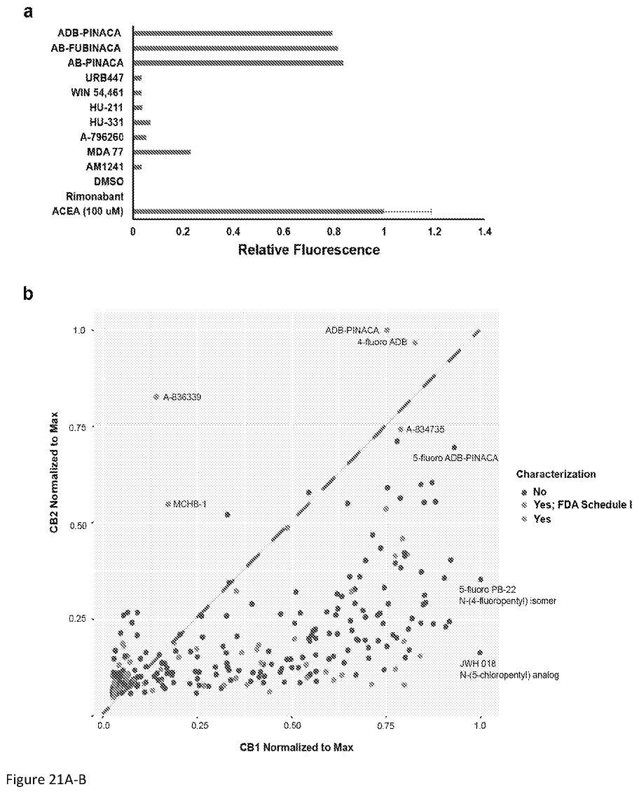

The biosensor screening assay showed that a large proportion of the compounds were active with the CB1R receptor relative to ACEA (FIG. 16A), and many compounds showed higher activity with CB2R than an endocannabinoid control, AEA (FIG. 16B). To validate the results, known and predicted agonists and antagonists of the receptors were examined individually. The known antagonist URB447 (LoVerne et al. 2009), predicted antagonist WIN54,461 (Eissenstat et al. 1995), non-binder HU-211 (Juttler et al. 2004), cannabidiol degradation product HU-311 (Mechoulam et al. 1968), or selective CB2R ligands A-796260 (Yao et al. 2008), AM-1241 (Yao et al. 2009), MDA 77 (Diaz et al. 2009), all showed less than 25% relative fluorescence of the ACEA positive control at CB1R at 10 nM (FIG. 21A), indicating that the yeast biosensors could readily identify both agonists and antagonists. In contrast, known agonists AB-FUBINACA, ADB-PINACA, and AB-PINACA showed 79-84% maximal activity at this concentration.

The CB1R biosensor gave consistent characterizations within compound classes. For example, in the PINACA agonist series (AB-PINACA, ADB-PINACA AND 5-fluoro ADB-PINACA isomer 2) all were shown to have EC₅₀ values in the low nanomolar range (FIG. 16C). Dose-response curves for AB-FUBINACA isomers were also obtained, including for many compounds that had previously been uncharacterized. AB-FUBINACA and AB-FUBINACA isomer 1 showed high maximal signaling, while isomers 2 and 5 had a lower response (FIG. 16D). Since AB-FUBINACA has been implicated in overdoses from recreational use along with a number of its variants, knowledge of the activity of structural variants could be useful for determination of compound scheduling.

For CB2R, there was again consistency within compound classes, and there were frequently structural cognates for activity. For example, structural analogues, such ADB-PINACA and AB-PINACA, showed high activity (Table 2), with the phenylmethyl group at the 1H-indazole and dimethylpropyl of ADB-BINACA being preferred by roughly 100-times over the pentyl and isopropyl, respectively, of AB-PINACA (FIG. 16E). ADB-BINACA had previously been uncharacterized against CB2R. The compound 4-fluoro ADB showed the greatest sensitivity amongst compounds tested (EC₅₀ of 1.35 nM, FIG. 16E), is known to be highly active at cannabinoid receptors, and is implicated in at least one death from overdose (Tokarczyk et al. 2022).

Similarly, the compounds A-836339 and A-834375 both have a tetramethylcyclopropylmethanone group adjacent to the carbonyl, which is known to confer CB2R bias and strong signaling (Frost et al. 2010), while A-834735 and 5-fluoro PB22 share the indole moieties common to many synthetic cannabinoids. A-836339 and A-834375 had EC_{50S} of 16.3 and 7.42 nM, respectively (FIG. 16F). Interestingly, the 5-fluoro PB-22 5-hydroxyquinoline isomer is an otherwise uncharacterized isomer of 5-fluoro PB-22 and showed robust signaling with an EC₅₀ of 216 nM (FIG. 16C).

### High-Throughput Drug Screening of Terpenes With Dual Cannabinoid Receptor Biosensor Strains

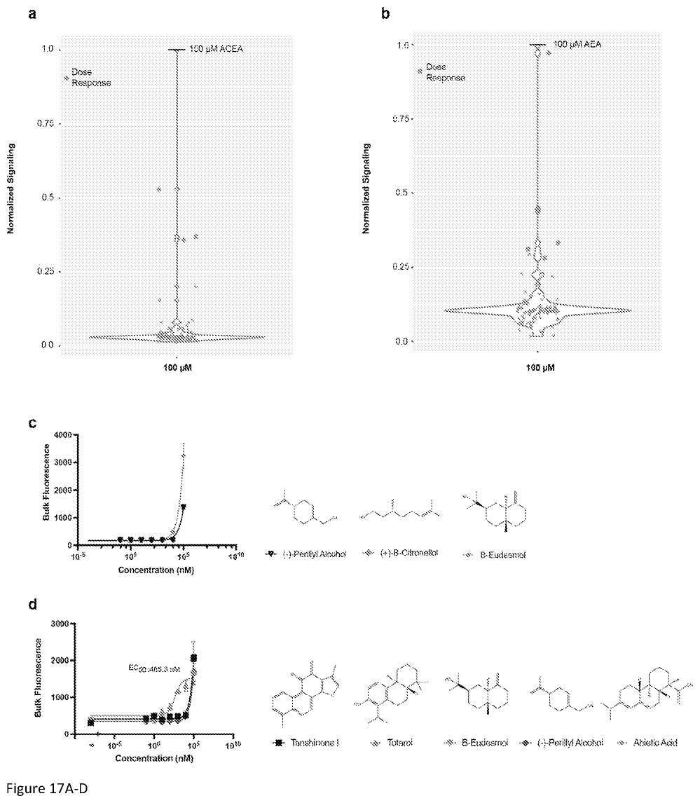

It has been hypothesized that THC and many of the terpenes found in cannabis strains work synergistically to create strain-variant effects (Russo et al., 2011) so a library of terpenes were assessed against both the CB1R and CB2R biosensor strains (FIG. 17A, B). A number of compounds in the library have been found in cannabis, and others are known to be bioactive (Russo et al. 2011). In order to detect even minor activities, compounds were screened at 100 μM, but very few compounds showed activity against either receptor. Amongst terpenes found to activate CB1R, both (+)-β-Citronellol and β-Eudesmol have previously been isolated from cannabis (Fletcher et al. 2019; Fischedick et al. 2015), and β-Eudesmol showed signaling at lower (10 μM) concentrations (FIG. 17C). Surprisingly, totatrol, isolated from the native New Zealand Podocarpus totara and more recently found in a wider range of plants (Sharp et al. 2001), was found to be active against CB2R at 10 nM, with an EC50 of 485 nM (FIG. 17D). While totarol has known therapeutic applications and antimicrobial properties (Gao et al. 2015; Kubo et al. 1992), it appears this is the first documented activity against CB2R.

### Results With Dual Cannabinoid Biosensor Strains Recapitulate and Extend Opportunities for Receptor-Specific Targeting

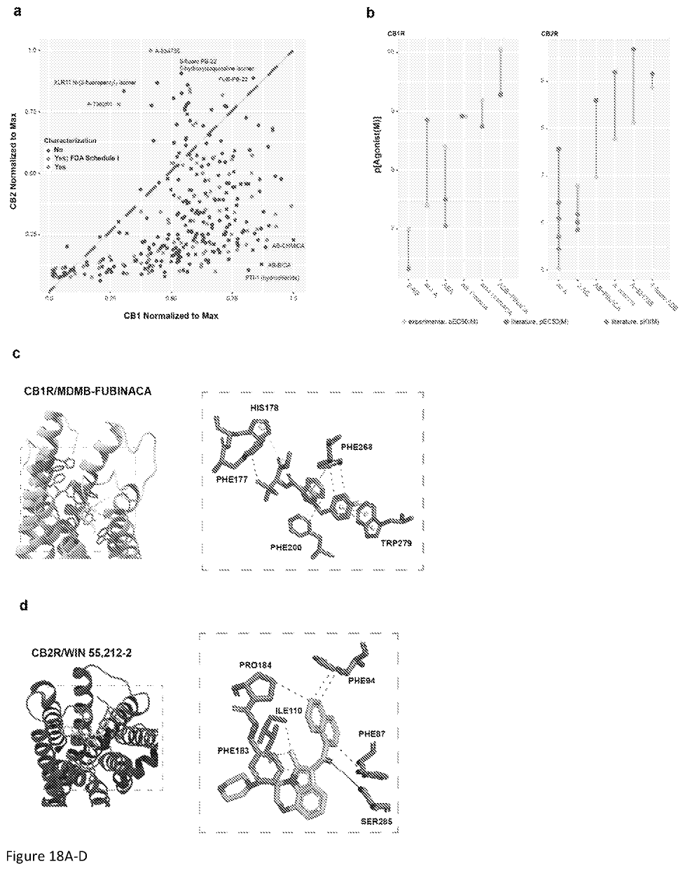

Because the binding pockets of CB1R and CB2R are so similar, it has historically proven challenging to find compounds that activate one receptor but not both. For example, the potent agonist 4-fluoro ADB shows high activity against both CB1R and CB2R (Cannaert et al. 2022; Lie et al. 2021). For the compounds tested with the dual cannabinoid biosensor strains, the overall overlap in binding and activation between receptors is also apparent (FIG. 18A, which synthesizes the CB1R and CB2R results with 1 μM compound from FIGS. 16A, B; analyses with 10 nM are in Supp. FIG. 17B). The ability of many compounds to activate or modulate CB1R recapitulates literature results, and is consistent with the fact that CB1R is able to accommodate a wider variety of ligands than CB2R, due to the greater flexibility of its binding site across receptor activity states.

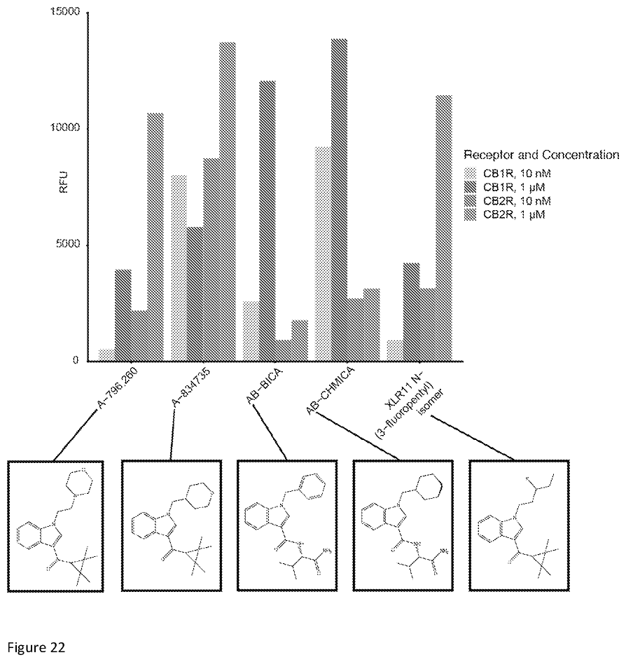

While the dual cannabinoid biosensor strains were largely consistent with the known literature, EC₅₀ and Kᵢ values were often higher than found in previous studies with mammalian cells (FIG. 22B). At least some of the differences observed may again be due to our observation that membrane composition can greatly impact receptor function: other groups have found evidence that cholesterol may act as a direct binding partner of endocannabinoids in the plasma membrane, and possibly have a role in membrane insertion of these compounds³², and thus the optimization of cholesterol levels may prove to be both receptor-and compound-specific. Nonetheless, there was a general concordance between the EC₅₀ values for AB-PINACA with CB1R in yeast and in mouse AtT-20 neuroblastoma cells (Banister et al., 2015) (both 1.2 nM), and for 4-fluoro ADB with CB2R in yeast (1.35 nM) and in (HEK) 293 T cells (0.69 nM, Lie et al. 2021), and overall general trends for modulation appeared the same between yeast-based and mammalian assays.

The ability to rapidly carry out assays with directly comparable results, coupled with structural analyses, now allows us to better identify receptor-specific compounds. The previously determined crystal structure of CB1R co-crystallized with the synthetic cannabinoid MDMB-FUBINACA (Kumar et all. 2019) indicates that the diazole ring (a shared feature between MDMB-FUBINACA and the AB-PINACA analogs) creates hydrophobic contacts with F200 and F268, and positions additional hydrophobic contacts with F174, F177, H178, and F200 (FIG. 18C) that lead to rearrangement of TM2 of CB1R during receptor activation. In consequence, the relative affinities of ADB-PINACA isomers (FIG. 18C; Table 2) and AB-FUBINACA isomers (FIG. 18D) can be rationalized, with the alkyl group adjacent to the amide bond differentially activating the receptor in several formats, including tert-butyl, 2-butyl, and isopropyl.

The high-throughput yeast-based assays also potentiate new forays into the identification of receptor-specific ligands. Amongst the CB1R agonists that were poor CB2R signalers (AB-BICA, AB-CHMICA and PTI-1), PTI-1 has a N1-indole pentyl side chain known to favor CB1R agonism, similar to AB-PINACA and ADB-PINACA (FIG. 16C) (Armenian et al. 2018). Interestingly, the uncharacterized fluoropentyl isomers of AB-PINACA roughly halved signaling with CB2R (Table 2), and further modulation of these moieties may lead to greater receptor discrimination. Discrimination via the amide-bonded moieties on synthetic cannabinoids (as described above) can also be rationalized by examining both structures and activities in the dual biosensor assay: AB-BICA is structurally similarly to ADB-BINACA, a strong CB2R agonist, but has a methylpropyl group adjacent to the amide rather than the dimethylpropyl of ADB-BINACA (FIG. 22), potentially indicating that CB1R favors the smaller carbon side group in this position.

Conversely, the drug A-796,260 was among the top CB2R targets that demonstrated low signaling with CB1R. This compound is a known CB2R-biased agonist whose 3-tetramethylcyclopropylmethanone group (in lieu of a 3-benzoyl or 3-naphthoyl group) confers CB2R bias. Both XLR11 N-(3-fluoropentyl) isomer and A-834735 are also CB2R-biased, and, like A-796,260, contain a 3-tetramethylcyclopropylmethanone group. This group proved important for CB2R signaling but precluded full CB1R activity-eight of the top twenty compounds at CB2R had this structural group, but none of the top twenty compounds at CB1R did (FIG. 18A). A previously determined crystal structure of CB2R co-crystallized with the synthetic cannabinoid WIN 55,212-2 highlights that the bulky 3-indole adducts on these various compounds trigger conformational changes in CB2R via the toggle switch W258 (Xing et al. 2020) (FIG. 18D). In a similar vein, some thirteen hydroxyquinoline isomers of PB-22 were screened with variable results across the receptors. Nitrogens at the 8th and 5th positions led to the greatest activity with CB2R, and dramatic decreases in activity were observed at the 3rd, 6th, and 7th positions (Table 3), further indicating that this may be a valuable new class of compounds where reactivities with CB2R could be finely tuned.

### Conclusions

The cannabinoid receptors CB1R and CB2R are of particular clinical interest for their roles in pain relief, appetite stimulation or suppression, and anti-epileptic properties. By engineering CB1R and the membrane environment of CB2R, dual biosensors were generated that could be used for the rapid screening of a wide variety of cannabinoids and other compounds, such as terpenes, including dozens of previously uncharacterized compounds. The results obtained with the yeast-based biosensors were congruent with those previously seen in mammalian cells, and further allowed the ready identification of known and new compounds with specificities for individual receptors. Based on functional and structural analyses PTI-1 has been identified as a potentially useful CB1R agonist, and a series of PB-22 isomers have been developed as CB2R agonists. Small structural modifications were found to greatly impact relative receptor specificity, with some PINACA modifications greatly reducing activity with

CB2R while leaving CB1R activity unafffected (Table 2). Similarly, some PB-22 hydroxyquinoline isomers favored CB1 by almost a factor of 3 (5-fluoro PB-22 4-hydroxyquinoline isomer; Table 3), while other closely related compounds favored CB2 by roughly 2-fold (5-fluoro PB-22 5-hydroxyisoquinoline isomer; Table 3).

In addition to advancing pharmaceutical drug development, the emerging cannabis industry exposes consumers to a large number of uncharacterized compounds. The sheer speed at which many psychoactive mixtures can be created makes it difficult for regulatory bodies and health authorities to keep pace, highlighting the need for studies that can quickly provide insights into receptor binding and specificity. The availability of facile comparative assays via the dual yeast biosensors builds on earlier work expressing CB2R in yeast and can provide a straightforward basis for comparison suitable for both research and regulatory organizations.

Finally, given that the complete biosynthesis of cannabinoids has been achieved in yeast, and that yeast biosensor strains expressing GPCRs have begun to show promise in detecting metabolites during bio-manufacturing (Ehrenwroth et al., 2017; Mukherjee et al. 2015), it is possible to envision adapting the GPCR-based sensors herein to the conjoined screening and selection of new pathways and receptors that are specific for any of a variety of cannabinoid and other compounds.

### Methods and Materials

### Molecular Biology

All cloning was performed using Golden Gate assembly following the MoClo Yeast toolkit (Lee et al. 2015 with some adaptations (see Parts list). Assemblies were performed as follows: 20 fmol part plasmids, 10,000 units Type IIs restriction enzymes (T7 DNA ligase, Esp31/BsaI-v2, NEB), and 1 μL T4 DNA ligase (NEB) in a 10 μL reaction. Thermal cycling was performed as follows: 1 minute 37° C., 2 minutes 16° C., for 25 cycles, then 37° C. for 30 minutes, and 80° C. for 10 minutes. 5 μL each reaction was transformed into 100 μL DH10B and transformed according to the Mix and Go Transformation kit (Zymo Research).

### Yeast Transformations

Yeast background strains were BY4741 (See yeast strain list). Yeast transformations were performed according to the EZ transformation II kit (Zymo research). 100 μL of cell prep was transformed with 1 μg or 5 μL of plasmid.

### Functional Assays

Yeast colonies were picked and grown overnight to saturation in pH 5.8 SD-His media in a 2.2 mL deep well plate (Axygen) grown in a plate-shaking incubator (30° C., 1000 rpm, 3 mm orbital). In the morning cultures were diluted 1:10 or 1:25 in SD-His media buffered to pH 7.1 with 100 mM MOPSO (unless otherwise specified). Ligands were added to the culture and incubated with shaking for 8 hours. Cells were then washed three times with ice-cold Tris buffer (pH 7) and diluted 1:20 for cytometry. All cytometry was performed on a Sony SA3800 spectral analyzer. Each sample was tested for 10,000 events, and read at a rate of 1,000 events per second. All assays as reported here were filtered only for singlet populations.

TABLE 2
Structure and activity of PINACA compounds.

| | Normalized |
|---|---|
| | Activity |
| Compound | at 1 μM |
| 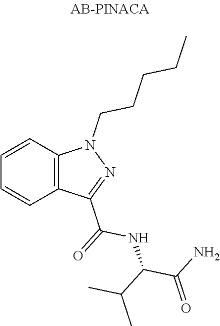 [SMILES: CCCCCn1nc(C(=O)N[C@H](C(N)=O)C(C)C)c2ccccc21] | CB1R: 0.550 CB2R: 0. [symbol] 33 |
| 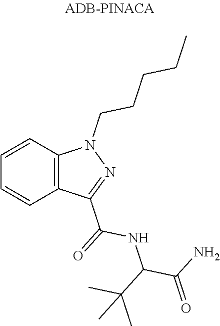 [SMILES: CCCCCn1nc(C(=O)NC(C(N)=O)C(C)(C)C)c2ccccc21] | CB1R: 0.590 CB2R: 0.700 |
| 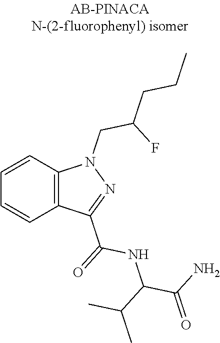 [SMILES: CCCC(F)Cn1nc(C(=O)NC(C(N)=O)C(C)C)c2ccccc21] | CB1R: 0.632 CB2R: 0.340 |
| 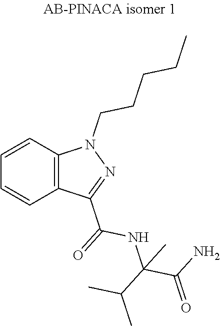 [SMILES: CCCCCn1nc(C(=O)NC(C)(C(N)=O)C(C)C)c2ccccc21] | CB1R: 0.810 CB2R: 0.303 |
| 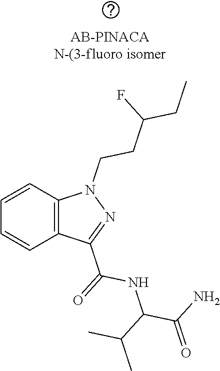 [SMILES: CCC(F)CCn1nc(C(=O)NC(C(N)=O)C(C)C)c2ccccc21] | CB1R: 0.700 CB2R: 0.310 |
| 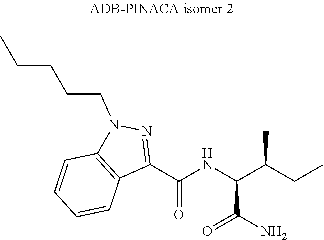 [SMILES: CCCCCn1nc(C(=O)N[C@H](C(N)=O)[C@@H](C)CC)c2ccccc21] | CB1R: 0.753 CB2R: 0.568 |
| 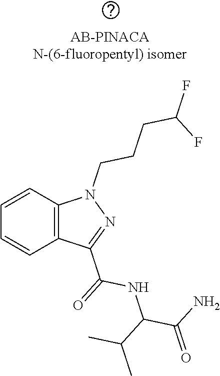 [SMILES: CC(C)C(NC(=O)c1nn(CCCC(F)F)c2ccccc12)C(N)=O] | CB1R: 0. [symbol] |
| 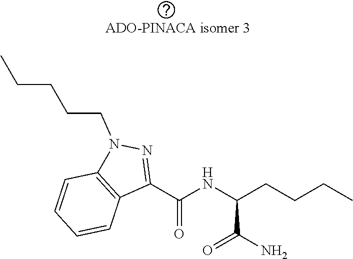 [SMILES: CCCCCn1nc(C(=O)N[C@@H](CCCC)C(N)=O)c2ccccc21] | CB1R: 0.735 CB2R: 0.460 |
| 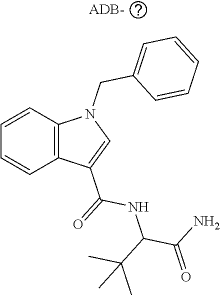 [SMILES: CC(C)(C)C(NC(=O)c1cn(Cc2ccccc2)c2ccccc12)C(N)=O] | CB1R: 0.842 CB2R: 0.255 |
| 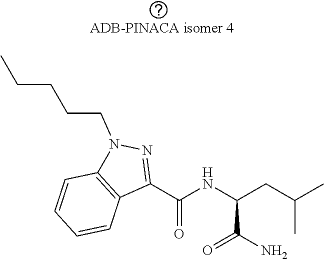 [SMILES: CCCCCn1nc(C(=O)N[C@@H](CC(C)C)C(N)=O)c2ccccc21] | CB1R: 0. [symbol] 71 CB2R: 0.5 [symbol] 1 |
| 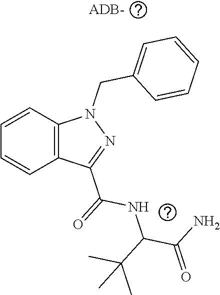 [SMILES: CC(C)(C)C(NC(=O)c1nn(Cc2ccccc2)c2ccccc12)C(N)=O] | CB1R: 0.91 [symbol] CB2R: 0.67 [symbol] |

[symbol]

indicates data missing or illegible when filed

TABLE 3
Structure and activity of 5-fluoro PB-22 compounds.

| | Normalized |
|---|---|
| | Activity |
| 5-fluoro PB-22 iso [symbol] | at 1 μm |
| 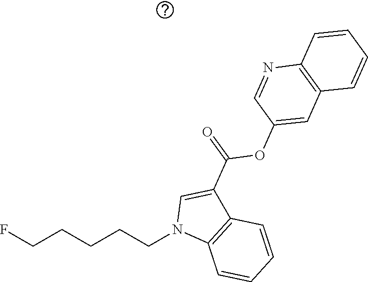 [SMILES: O=C(Oc1cnc2ccccc2c1)c1cn(CCCCCF)c2ccccc12] | CB1R: 0.530 CB2R: 0.14 [symbol] |
| 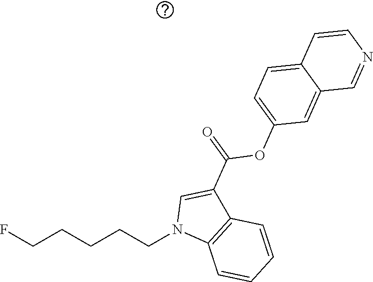 [SMILES: O=C(Oc1ccc2ccncc2c1)c1cn(CCCCCF)c2ccccc12] | CB1R: 0.5 [symbol] 0 CB2R: 0.339 |
| 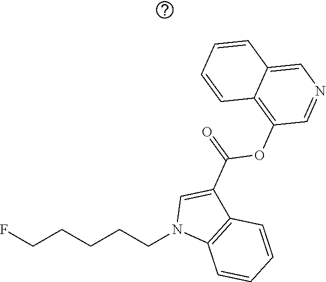 [SMILES: O=C(Oc1cncc2ccccc12)c1cn(CCCCCF)c2ccccc12] | CB1R: 0.570 CB2R: 0.78 [symbol] |
| 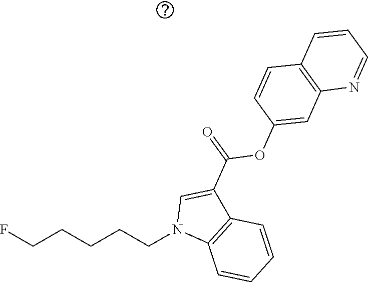 [SMILES: O=C(Oc1ccc2cccnc2c1)c1cn(CCCCCF)c2ccccc12] | CB1R: 0.550 CB2R: 0.1 [symbol] |
| 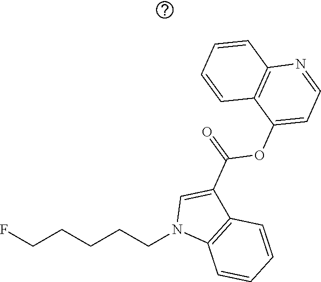 [SMILES: O=C(Oc1ccnc2ccccc12)c1cn(CCCCCF)c2ccccc12] | CB1R: 0.829 CB2R: 0.247 |
| 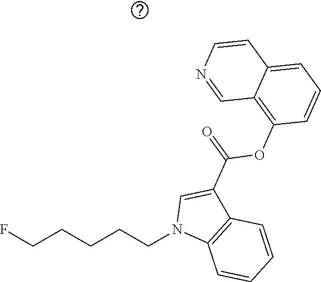 [SMILES: O=C(Oc1cccc2ccncc12)c1cn(CCCCCF)c2ccccc12] | CB1R: 0.5 [symbol] 0 CB2R: 0.771 |
| 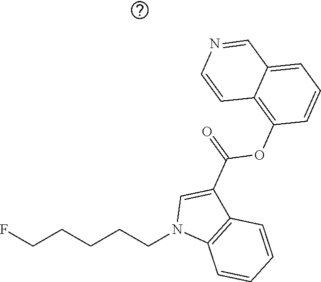 [SMILES: O=C(Oc1cccc2cnccc12)c1cn(CCCCCF)c2ccccc12] | CB1R: 0.5 [symbol] 0 CB2R: 0.90 [symbol] |
| 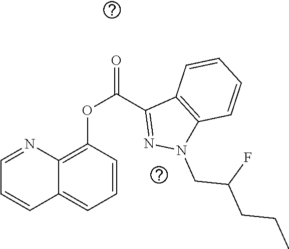 [SMILES: CCCC(F)Cn1nc(C(=O)Oc2cccc3cccnc23)c2ccccc21] | CB1R: 0.531 CB2R: 0.582 |
| 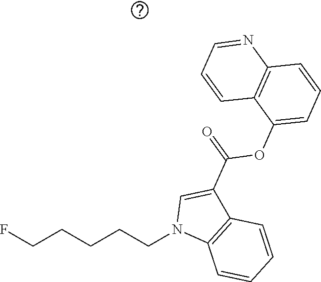 [SMILES: O=C(Oc1cccc2ncccc12)c1cn(CCCCCF)c2ccccc12] | CB1R: 0. [symbol] 04 CB2R: 0.4 [symbol] 8 |
| 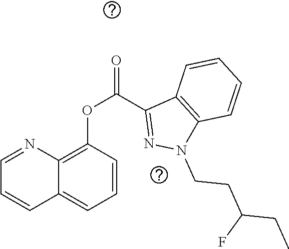 [SMILES: CCC(F)CCn1nc(C(=O)Oc2cccc3cccnc23)c2ccccc21] | CB1R: 0. [symbol] 30 CB2R: 0.5 [symbol] 2 |
| 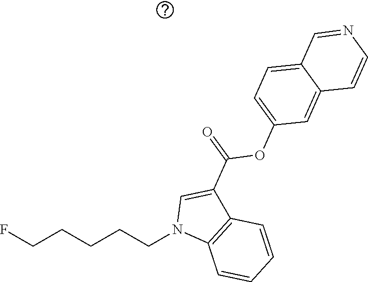 [SMILES: O=C(Oc1ccc2cnccc2c1)c1cn(CCCCCF)c2ccccc12] | CB1R: 0. [symbol] 26 CB2R: 0.331 |
| 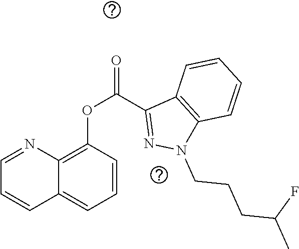 [SMILES: CC(F)CCCn1nc(C(=O)Oc2cccc3cccnc23)c2ccccc21] | CB1R: 0.5 [symbol] 0 CB2R: 0. [symbol] 36 |
| 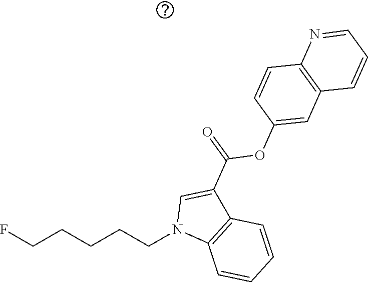 [SMILES: O=C(Oc1ccc2ncccc2c1)c1cn(CCCCCF)c2ccccc12] | CB1R: 0.500 CB2R: 0.141 |

[symbol]

indicates data missing or illegible when filed

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

1. Hauser, A. S., Attwood, M. M., Rask-Andersen, M., Schioth, H. B. & Gloriam, D. E. Trends in GPCR drug discovery: new agents, targets and indications. Nat. Rev. Drug Discov. 16, 829-842 (2017).
2. Versele, M., Lemaire, K. & Thevelein, J. M. Sex and sugar in yeast: two distinct GPCR systems. EMBO Rep. 2, 574-579 (2001).
3. Brown, A. et al. Functional coupling of mammalian receptors to the yeast mating pathway using novel yeast/mammalian G protein alpha-subunit chimeras. Yeast 16, 11-22 (2000).
4. Irving, A. J., McDonald, N. A. & Harkany, T. CB1 Cannabinoid Receptors: Molecular Biology, Second Messenger Coupling and Polarized Trafficking in Neurons. in Cannabinoids and the Brain (ed. Köfalvi, A.) 59-73 (Springer US, 2008). doi: 10.1007/978-0-387-74349-3_5.
5. Zou, S. & Kumar, U. Cannabinoid Receptors and the Endocannabinoid System: Signaling and Function in the Central Nervous System. Int. J. Mol. Sci. 19, 833 (2018).
6. Zou, S. & Kumar, U. Cannabinoid Receptors and the Endocannabinoid System: Signaling and Function in the Central Nervous System. Int. J. Mol. Sci. 19, 833 (2018).
7. Lu, H.-C. & Mackie, K. An introduction to the endogenous cannabinoid system. Biol. Psychiatry 79, 516-525 (2016).
8. Ranganathan, M. et al. Reduced Brain Cannabinoid Receptor Availability In Schizophrenia. Biol. Psychiatry 79, 997-1005 (2016).
9. Turcotte, C., Blanchet, M.-R., Laviolette, M. & Flamand, N. The CB2 receptor and its role as a regulator of inflammation. Cell. Mol. Life Sci. 73, 4449-4470 (2016).
10. Tai, S. & Fantegrossi, W. E. Synthetic Cannabinoids: Pharmacology, Behavioral Effects, and Abuse Potential. Curr. Addict. Rep. 1, 129-136 (2014).
11. Adams, A. J. et al. “Zombie” Outbreak Caused by the Synthetic Cannabinoid AMB-FUBINACA in New York. N. Engl. J. Med. 376, 235-242 (2017).
12. Lobato-Freitas, C. et al. Overview of Synthetic Cannabinoids ADB-FUBINACA and AMB-FUBINACA: Clinical, Analytical, and Forensic Implications. Pharmaceuticals 14, 186 (2021).
13. Tokarczyk, B., Jurczyk, A., Krupińska, J. & Adamowicz, P. Fatal intoxication with new synthetic cannabinoids 5F-MDMB-PICA and 4F-MDMB-BINACA—parent compounds and metabolite identification in blood, urine and cerebrospinal fluid. Forensic Sci. Med. Pathol. 1-10 (2022) doi: 10.1007/s12024-022-00492-3.
14. Hua, T. et al. Activation and Signaling Mechanism Revealed by Cannabinoid Receptor-Gi Complex Structures. Cell 180, 655-665.e18 (2020).
15. Lengger, B. & Jensen, M. K. Engineering G protein-coupled receptor signalling in yeast for biotechnological and medical purposes. FEMS Yeast Res. 20, foz087 (2019).
16. Lee, M. E., DeLoache, W. C., Cervantes, B. & Dueber, J. E. A Highly Characterized Yeast Toolkit for Modular, Multipart Assembly. ACS Synth. Biol. 4, 975-986 (2015).
17. Membrane Assembly of the Cannabinoid Receptor 1: Impact of a Long N-Terminal Tail|Molecular Pharmacology. https://molpharm.aspetjournals.org/content/64/3/570.
18. Hebert-Chatelain, E. et al. A cannabinoid link between mitochondria and memory. Nature 539, 555-559 (2016).
19. Smith, M. et al. The Effects of the Endocannabinoids Anandamide and 2-Arachidonoylglycerol on Human Osteoblast Proliferation and Differentiation. PLOS ONE 10, e0136546 (2015).
20. Miettinen, K. et al. A GPCR-based yeast biosensor for biomedical, biotechnological, and point-of-use cannabinoid determination. Nat. Commun. 13, 3664 (2022).
21. Shaw, W. M. et al. Screening microbially produced 49-tetrahydrocannabinol using a yeast biosensor workflow. Nat. Commun. 13, 5509 (2022).
22. Gonsiorek, W. et al. Endocannabinoid 2-Arachidonyl Glycerol Is a Full Agonist through Human Type 2 Cannabinoid Receptor: Antagonism by Anandamide. Mol. Pharmacol. 57, 1045-1050 (2000).
23. Ishii, J. & Kondo, A. Cell Wall Trapping of Autocrine Peptides for Human G-Protein-Coupled Receptors on the Yeast Cell Surface. PLOS One 7, (2012).
24. Bean, B. D. M. et al. Functional expression of opioid receptors and other human GPCRs in yeast engineered to produce human sterols. Nat. Commun. 13, 2882 (2022).
25. Kapolka, N. J. et al. DCyFIR: a high-throughput CRISPR platform for multiplexed G protein-coupled receptor profiling and ligand discovery. Proc. Natl. Acad. Sci. 117, 13117-13126 (2020).
26. Lengger, B. et al. Serotonin G Protein-Coupled Receptor-Based Biosensing Modalities in Yeast. ACS Sens. 7, 1323-1335 (2022).
27. Gimpl, G. Interaction of G protein coupled receptors and cholesterol. Chem. Phys. Lipids 199, 61-73 (2016).
28. Sarkar, P. & Chattopadhyay, A. Cholesterol in GPCR Structures: Prevalence and Relevance. J. Membr. Biol. 255, 99-106 (2022).
29. Oddi, S. et al. Functional characterization of putative cholesterol binding sequence (CRAC) in human type-1 cannabinoid receptor. J. Neurochem. 116, 858-865 (2011).
30. Bari, M. et al. Effect of Lipid Rafts on Cb2 Receptor Signaling and 2-Arachidonoyl-Glycerol Metabolism in Human Immune Cells. J. Immunol. 177, 4971-4980 (2006).
31. Yeliseev, A. et al. Cholesterol as a modulator of cannabinoid receptor CB2 signaling. Sci. Rep. 11, 3706 (2021).
32. Scala, C. D., Fantini, J., Yahi, N., Barrantes, F. J. & Chahinian, H. Anandamide Revisited: How Cholesterol and Ceramides Control Receptor-Dependent and Receptor-Independent Signal Transmission Pathways of a Lipid Neurotransmitter. Biomolecules 8, 31 (2018).
33. LoVerme, J. et al. Synthesis and characterization of a peripherally restricted CB1 cannabinoid antagonist, URB447, that reduces feeding and body-weight gain in mice. Bioorg. Med. Chem. Lett. 19, 639-643 (2009).
34. Eissenstat, M. A. et al. Aminoalkylindoles: structure-activity relationships of novel cannabinoid mimetics. J. Med. Chem. 38, 3094-3105 (1995).
35. Jüttler, E. et al. The cannabinoid dexanabinol is an inhibitor of the nuclear factor-kappa B (NF-κB). Neuropharmacology 47, 580-592 (2004).
36. Mechoulam, R., Ben-Zvi, Z. & Gaoni, Y. Hashish—XIII: On the nature of the beam test. Tetrahedron 24, 5615-5624 (1968).
37. Yao, B. B. et al. In vitro and in vivo characterization of A-796260: a selective cannabinoid CB2 receptor agonist exhibiting analgesic activity in rodent pain models. Br. J. Pharmacol. 153, 390-401 (2008).
38. Yao, B. B. et al. In vitro pharmacological characterization of AM1241: a protean agonist at the cannabinoid CB2 receptor? Br. J. Pharmacol. 149, 145-154 (2006).
39. Diaz, P. et al. 6-Methoxy-N-alkyl isatin acylhydrazone derivatives as a novel series of potent selective cannabinoid receptor 2 inverse agonists: design, synthesis, and binding mode prediction. J. Med. Chem. 52, 433-444 (2009).
40. Frost, J. M. et al. Indol-3-ylcycloalkyl Ketones: Effects of N1 Substituted Indole Side Chain Variations on CB2 Cannabinoid Receptor Activity. J. Med. Chem. 53, 295-315 (2010).
41. Russo, E. B. Taming THC: potential cannabis synergy and phytocannabinoid-terpenoid entourage effects. Br. J. Pharmacol. 163, 1344-1364 (2011).
42. Fletcher, R. S. & Mckay, J. United States Patent: 10499584—Industrial hemp Cannabis cultivars and seeds with stable cannabinoid profiles. (2019).
43. Fischedick J, E. S. Cannabinoids and Terpenes as Chemotaxonomic Markers in Cannabis. Nat. Prod. Chem. Res. 03, (2015).
44. Sharp, H. et al. Totarol, totaradiol and ferruginol: three diterpenes from Thuja plicata (Cupressaceae). Biochem. Syst. Ecol. 29, 215-217 (2001).
45. Gao, Y. et al. Totarol prevents neuronal injury in vitro and ameliorates brain ischemic stroke: Potential roles of Akt activation and HO-1 induction. Toxicol. Appl. Pharmacol. 289, 142-154 (2015).
46. Kubo, I., Muroi, H. & Himejima, M. Antibacterial Activity of Totarol and Its Potentiation. J. Nat. Prod. 55, 1436-1440 (1992).
47. Cannaert, A. et al. Synthesis and in Vitro Cannabinoid Receptor 1 Activity of Recently Detected Synthetic Cannabinoids 4F-MDMB-BICA, 5F-MPP-PICA, MMB-4en-PICA, CUMYL-CBMICA, ADB-BINACA, APP-BINACA, 4F-MDMB-BINACA, MDMB-4en-PINACA, A-CHMINACA, 5F-AB-P7AICA, 5F-MDMB-P7AICA, and 5F-AP7AICA. ACS Chem. Neurosci. 11, 4434-4446 (2020).
48. Lie, W. et al. Diagnosing intake and rationalizing toxicities associated with 5F-MDMB-PINACA and 4F-MDMB-BINACA abuse. Arch. Toxicol. 95, 489-508 (2021).
49. Banister, S. D. et al. Pharmacology of Indole and Indazole Synthetic Cannabinoid Designer Drugs AB-FUBINACA, ADB-FUBINACA, AB-PINACA, ADB-PINACA, 5F-AB-PINACA, 5F-ADB-PINACA, ADBICA, and 5F-ADBICA. ACS Chem. Neurosci. 6, 1546-1559 (2015).
50. Kumar, K. K. et al. Structure of a Signaling Cannabinoid Receptor 1-G Protein Complex. Cell 176, 448-458.e12 (2019).
51. Armenian, P. et al. Intoxication from the novel synthetic cannabinoids AB-PINACA and ADB-PINACA: A case series and review of the literature. Neuropharmacology 134, 82-91 (2018).
52. Xing, C. et al. Cryo-EM Structure of the Human Cannabinoid Receptor CB2-Gi Signaling Complex. Cell 180, 645-654.e13 (2020).
53. Luo, X. et al. Complete biosynthesis of cannabinoids and their unnatural analogues in yeast. Nature 567, 123-126 (2019).
54. Ehrenworth, A. M., Claiborne, T. & Peralta-Yahya, P. Medium-Throughput Screen of Microbially Produced Serotonin via a G-Protein-Coupled Receptor-Based Sensor. ACS Publications https://pubs.acs.org/doi/pdf/10.1021/acs.biochem.7b00605 (2017) doi: 10.1021/acs.biochem.7b00605.
55. Mukherjee, K., Bhattacharyya, S. & Peralta-Yahya, P. GPCR-Based Chemical Biosensors for Medium-Chain Fatty Acids. ACS Synth. Biol. 4, 1261-1269 (2015).
56. Griffin, A., Anvar, M., Hamling, K. & Baraban, S. C. Phenotype-Based Screening of Synthetic Cannabinoids in a Dravet Syndrome Zebrafish Model. Front. Pharmacol. 11, (2020).
57. Raup-Konsavage, W. M. et al. Synthetic Cannabinoid Activity Against Colorectal Cancer Cells. Cannabis Cannabinoid Res. 3, 272-281 (2018).
58. Chin, C.-L. et al. Differential effects of cannabinoid receptor agonists on regional brain activity using pharmacological MRI. Br. J. Pharmacol. 153, 367-379 (2008).
59. Hillard, C. J. et al. Synthesis and Characterization of Potent and Selective Agonists of the Neuronal Cannabinoid Receptor (CB1). J. Pharmacol. Exp. Ther. 289, 1427-1433 (1999).
60. Yao, B. B. et al. Characterization of a Cannabinoid CB2 Receptor-Selective Agonist, A-836339 [2,2,3,3-Tetramethyl-cyclopropanecarboxylic Acid [3-(2-Methoxy-ethyl)-4,5-dimethyl-3H-thiazol-(2Z)-ylidene]-amide], Using in Vitro Pharmacological Assays, in Vivo Pain Models, and Pharmacological Magnetic Resonance Imaging. J. Pharmacol. Exp. Ther. 328, 141-151 (2009).
61. Ben-Shabat, S. et al. An entourage effect: inactive endogenous fatty acid glycerol esters enhance 2-arachidonoyl-glycerol cannabinoid activity. Eur. J. Pharmacol. 353, 23-31 (1998).
62. Mechoulam, R. et al. Identification of an endogenous 2-monoglyceride, present in canine gut, that binds to cannabinoid receptors. Biochem. Pharmacol. 50, 83-90 (1995).
63. Adasme, M. F. et al. PLIP 2021: expanding the scope of the protein-ligand interaction profiler to DNA and RNA. Nucleic Acids Res. 49, W530-W534 (2021).
64. Southan, C. et al. The IUPHAR/BPS Guide to PHARMACOLOGY in 2016: towards curated quantitative interactions between 1300 protein targets and 6000 ligands. Nucleic Acids Res 44, D1054-D1068 (2016).
65. Shaw, W. M. et al. Engineering a Model Cell for Rational Tuning of GPCR Signaling. Cell 177, 782-796.e27 (2019).
66. Yasi, E. A. & Peralta-Yahya, P. Screening for Serotonin Receptor 4 Agonists Using a GPCR-Based Sensor in Yeast. in G Protein-Coupled Receptor Screening Assays: Methods and Protocols (eds. Martins, S. A. M. & Prazeres, D. M. F.) 77-84 (Springer US, 2021). doi: 10.1007/978-1-0716-1221-75.
67. Lagane, B. et al. Role of sterols in modulating the human mu-opioid receptor function in Saccharomyces cerevisiae. J Biol Chem 275, 33197-33200 (2000).
68. Andersson, H., D′Antona, A. M., Kendall, D. A., Heijne, G. V. & Chin, C.-N. Membrane Assembly of the Cannabinoid Receptor 1: Impact of a Long N-Terminal Tail. Mol Pharmacol 64, 570-577 (2003).
69. Hung, W.-C. et al. Comparative Study of the Condensing Effects of Ergosterol and Cholesterol. Biophys J 110, 2026-2033 (2016).
70. Patel, M., Finlay, D. B. & Glass, M. Biased agonism at the cannabinoid receptors—Evidence from synthetic cannabinoid receptor agonists. Cell Signal 78, 109865 (2021).
71. Diez-Alarcia, R. et al. Biased Agonism of Three Different Cannabinoid Receptor Agonists in Mouse Brain Cortex. Front Pharmacol 7, 415 (2016).
72. Ostrov, N. et al. A modular yeast biosensor for low-cost point-of-care pathogen detection. Science Advances 3, e1603221.

1. A method of engineering a eukaryotic cell to express a heterologous G protein coupled receptor (GPCR), wherein said GPCR is a cannabinoid receptor, the method comprising:
a. Replacing a gene encoding native Ste2 and/or Ste3 of the eukaryotic cell with a gene encoding a heterologous GPCR, wherein said heterologous GPCR has a truncated N-terminus of at least 5 residues; and
b. Replacing a gene encoding native G alpha protein (Gpa1) of the eukaryotic cell with a gene encoding a chimeric Gpa1.

2. The method of claim 1, wherein the eukaryotic cell is a yeast cell.

3. The method of claim 1, wherein the cannabinoid receptor is human cannabinoid receptor type I (CB1R).

4. The method of claim 1, wherein the chimeric Gpa1 comprises at least 3 amino acids from a human G-alpha variant.

5-6. (canceled)

7. The method of claim 1, wherein one or more genes that encode other cellular functions or responses are disabled or replaced.

8. The method of claim 7, wherein a gene encoding Sst2 is knocked out.

9. The method of claim 7, wherein cell cycle arrest genes are knocked out.

10. The method of claim 9, wherein the cell cycle arrest gene which is knocked out encodes Far1.

11. The method of claim 1, wherein the eukaryotic cell is further engineered to express a reporter of exogenous GPCR expression.

12. The method of claim 11, wherein pheromone-inducible Fig1 gene is replaced.

13. (canceled)

14. The method of claim 1, wherein the cell is further engineered to express one or more pre-pro signals and/or signal sequences along with GPCR.

15. The method of claim 14, wherein the pre-pro signal is syn-prepro.

16. The method of claim 1, wherein the cell is further engineered with a heterologous promoter of the GPCR.

17. The method of claim 1, wherein CRISPR/Cas9 is used to perform engineering.

18. The method of claim 7, wherein gene knockout is done by in-frame stop codon followed by a barcode.

19-20. (canceled)

21. An engineered eukaryotic cell which expresses a heterologous G protein coupled receptor (GPCR), wherein native Ste2 and/or Ste3 have been replaced with a heterologous GPCR, wherein said heterologous GPCR has a truncated N terminus of at least 5 residues; and further wherein native G alpha protein (Gpa1) has been replaced with a gene encoding a chimeric Gpa1.

22-39. (canceled)

40. A biosensor comprising the engineered eukaryotic cell of claim 21.

41. A method of identifying a compound capable of binding to a non-naturally occurring GPCR, the method comprising:
a. exposing a test compound to an engineered eukaryotic cell which expresses a heterologous G protein coupled receptor (GPCR), wherein native Ste2 and/or Ste3 have been replaced with a heterologous GPCR, wherein said heterologous GPCR has a truncated N terminus of at least 5 residues; and further wherein native G alpha protein (Gpa1) has been replaced with a gene encoding a chimeric Gpa1;
b. evaluating whether the test compound binds to the GPCR.

42-71. (canceled)

72. A method of determining relative binding of a compound to CB1R and CB2R, the method comprising:
a. exposing a test compound to both CB1R and CB2R;
b. evaluating relative binding of the compound to each of CB1R and CB2R; and
c. determining whether preferential binding occurs to CB1R or CB2R.

73-84. (canceled)

85. A biosensor for the detection of compounds which interact with CB1R, CB2R, or both, wherein the biosensor comprises both CB1R and CB2R.

86-90. (canceled)
